SEQUENCE LISTING (13 sequences)

SEQ ID NO: 1 - protein, 99 aa, Human MCP-1
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 2 - protein, 76 aa, Pan troglodytes (chimpanzee) MCP-1
QPDAINAPVTCCYNFTNRKISVQRLASYRRITSSKCPKEAVIFKTIVAKEICADPKQKWVQDSMDHLDKQTQTPKT

SEQ ID NO: 3 - protein, 76 aa, Pongo abelii (Sumatran orang-utan) MCP-1
QPDAINAPVTCCYNFTNRKISVQRLASYRRITSSKCPKEAVIFKTIVAKEICADPKQKWVQDSMDHLDKQTQTLKT

SEQ ID NO: 4 - protein, 76 aa, Macaca fascicularis (Crab eating macaque) MCP-1
QPDAINAPVTCCYNFTNRKISVQRLASYRRITSSKCPKEAVIFKTIVAKEICADPKQKWVQDSMDHLDKQIQTPKP

SEQ ID NO: 5 - protein, 78 aa, Canis familiaris MCP-1
QPDAIISPVTCCYTLTNKKISIQRLASYKRVTSSKCPKEAVIFKTVLNKEICADPKQKWVQDSMAHLDKKSQTQTAKP

SEQ ID NO: 6 - protein, 76 aa, Sus scrofa MCP-1
QPDAINSPVTCCYTLTSKKISMQRLMSYRRVTSSKCPKEAVIFKTIAGKEICAEPKQKWVQDSISHLDKKNQTPKP

SEQ ID NO: 7 - protein, 76 aa, Bos taurus MCP-1
QPDAINSQVACCYTFNSKKISMQRLMNYRRVTSSKCPKEAVIFKTILGKELCADPKQKWVQDSINYLNKKNQTPKP

SEQ ID NO: 8 - protein, 76 aa, Equus caballus MCP-1
QPDAINSPVTCCYTFTGKKISSQRLGSYKRVTSSKCPKEAVIFKTILAKEICADPEQKWVQDAVKQLDKKAQTPKP

SEQ ID NO: 9 - protein, 125 aa, Mus musculus MCP-1
QPDAVNAPLTCCYSFTSKMIPMSRLESYKRITSSRCPKEAVVFVTKLKREVCADPKKEWVQTYIKNLDRNQMRSEPTTLFKTASALRSSAPLNVKLTRKSEANASTTFSTTTSSTSVGVTSVTVN

SEQ ID NO: 10 - protein, 125 aa, Rattus norvegicus MCP-1
QPDAVNAPLTCCYSFTGKMIPMSRLENYKRITSSRCPKEAVVFVTKLKREICADPNKEWVQKYIRKLDQNQVRSETTVFYKIASTLRTSAPLNVNLTHKSEANASTLFSTTTSSTSVEVTSMTEN

SEQ ID NO: 11 - protein, 99 aa, Human MCP-2
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 12 - protein, 99 aa, Human MCP-3
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 13 - protein, 98 aa, Human MCP-4
MKVSAVLLCLLLMTAAFNPQGLAQPDALNVPSTCCFTFSSKKISLQRLKSYVITTSRCPQKAVIFRTKLGKEICADPKEKWVQNYMKHLGRKAHTLKT

(19) United States
(12) Patent Application Publication
(10) Pub. No.: US 2011/0212853 A1
(43) Pub. Date: Sep. 1, 2011

(54) NOVEL DIAGNOSTIC METHOD

(75) Inventors: Holger Cynis, Halle/Saale (DE); Martin Kleinschmidt, Halle/Saale (DE); Kathrin Gans, Halle/Saale (DE); Jens-Ulrich Rahfeld, OT Roeblingen am See (DE); Hans-Ulrich Demuth, Halle/Saale (DE); Nadine Taudte, Halle/Saale (DE)

(73) Assignee: PROBIODRUG AG, Halle/Saale (DE)

(21) Appl. No.: 13/030,258

(22) Filed: Feb. 18, 2011

Related U.S. Application Data
(60) Provisional application No. 61/305,721, filed on Feb. 18, 2010.

(51) Int. Cl. C40B 30/04; C12Q 1/02; C40B 30/00; G01N 33/566

(52) U.S. Cl. 506/9; 435/7.9; 435/7.93; 435/7.94; 435/7.95; 435/7.92; 435/29; 506/7; 435/7.21; 436/501

(57) ABSTRACT

A method of monitoring treatment of an inflammatory disease or an inflammatory associated disease with the use of the ratio of N-terminal pyroglutamate modified MCP-1 (MCP-1 N1pE):total concentration of MCP-1 within a biological sample as a biomarker, and a method for determining the proportion of N-terminal pyroglutamate modified MCP-1 in relation to the total concentration of MCP-1 in biological samples. Also disclosed are a diagnostic kit and a method for screening a glutaminyl cyclase (QC) inhibitor or measuring the effectiveness of a glutaminyl cyclase (QC) inhibitor.

### CROSS-REFERENCE TO RELATED APPLICATIONS

This application claims is the non-provisional of claims the benefit of priority to U.S. Provisional Application Ser. No. 61/305,721, filed on Feb. 18, 2010, which is incorporated herein by reference in its entirety.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present disclosure relates to a method to monitor treatment of an inflammatory disease or an inflammatory associated disease with the use of the ratio of N-terminal pyroglutamate modified MCP-1 (MCP-1 N1pE):total concentration of MCP-1 within a biological sample as a biomarker and further concerns a novel method to determine the proportion of N-terminal pyroglutamate modified MCP-1 in relation to the total concentration of MCP-1 in biological samples. The present disclosure also provides a diagnostic kit and a method for screening a glutaminyl cyclase (QC) inhibitor or measuring the effectiveness of a glutaminyl cyclase (QC) inhibitor.

### BACKGROUND OF THE INVENTION

Chemotactic cytokines (chemokines) are proteins that attract and activate leukocytes and are thought to play a fundamental role in inflammation. Chemokines are divided into four families categorized by the appearance of N-terminal cysteine residues (C-; CC-; CXC- and CX3C-chemokines). CC-chemokines (alias β-chemokines) attract preferentially monocytes to sites of inflammation. Monocyte infiltration is considered to be a key event in a number of disease conditions (Gerard, C. and Rollins, B. J. (2001) Nat. Immunol 2, 108-115; Bhatia, M., et al., (2005) Pancreatology. 5, 132-144; Kitamoto, S., Egashira, K., and Takeshita, A. (2003) J Pharmacol Sci. 91, 192-196).

MCP-1 (monocyte chemotactic protein-1, CCL2) is a member of the CC-family of chemokines. In this family, the 2 cysteines nearest to the amino terminus are adjacent to each other (thus C-C proteins). As with many other CC chemokines, the MCP-1 gene is located on chromosome 17 in humans. The cell surface receptors that bind MCP-1 are CCR2 and CCR5.

Four different human MCPs have been discovered: MCP-1 (CCL2), MCP-2 (CCL8), MCP-3 (CCL7) and MCP-4 (CCL13). The MCPs may be considered as a sub-family of the CC chemokines. All MCPs display a preference for attracting monocytes but show differences in their expression levels and chemotactic potential (Luini, W., et al., (1994) Cytokine 6, 28-31; Uguccioni, M., et al., (1995) Eur J Immunol 25, 64-68) Berkhout, et al., (1997) JBC.

In the following, the amino acid sequence of human MCP-1 is indicated:

Human MCP-1 (CCL2) (UniProtKB/Swiss-Prot P13500)
Protein (Signal Sequence in bold: 23 aa;
Mature MCP-1: 76 aa)
SEQ ID NO: 1
MKVSAALLCLLLIAATFIPQGLAQPDAINAPVTCCYNFTNRKISVQRLAS
YRRITSSKCPKEAVIFKTIVAKEICADPKQKWVQDSMDHLDKQTQTPKT

Consistent with it being a member of the chemokine β family, MCP-1 has been shown to chemoattract and activate monocytes in vitro at subnanomolar concentrations. Elevated MCP-1 expression has been detected in a variety of pathologic conditions that involve monocyte accumulation and activation, including a number of inflammatory and non-inflammatory disease states, like rheumatoid arthritis, atherosclerosis, asthma, obesity and delayed hypersensitivity reactions.

A number of studies have underlined in particular the crucial role of MCP-1 for the development of atherosclerosis (Gu, L., et al., (1998) Mol. Cell 2, 275-281; Gosling, J., et al., (1999) J Clin. Invest 103, 773-778); rheumatoid arthritis (Gong, J. H., et al., (1997) J Exp. Med 186, 131-137; Ogata, H., et al., (1997) J Pathol. 182, 106-114); pancreatitis (Bhatia, M., et al., (2005) Am. J Physiol Gastrointest. Liver Physiol 288, G1259-G1265); Alzheimer's disease (Yamamoto, M., et al., (2005) Am. J Pathol. 166, 1475-1485); lung fibrosis (Inoshima, I., et al., (2004) Am. J Physiol Lung Cell Mol. Physiol 286, L1038-L1044); renal fibrosis (Wada, T., et al., (2004) J Am. Soc. Nephrol. 15, 940-948), and graft rejection (Saiura, A., et al., (2004) Arterioscler. Thromb. Vasc. Biol. 24, 1886-1890). Furthermore, MCP-1 might also play a role in gestosis (Katabuchi, H., et al., (2003) Med Electron Microsc. 36, 253-262), contribute to pathologies associated with hyperinsulinemia and obesity, including type II diabetes (Sartipy, P. and Loskutoff, P. J. (2003) Proc. Natl. Acad. Sci. U.S.A 100, 7265-70), as a paracrine factor in tumor development (Ohta, M., et al., (2003) Int. J Oncol. 22, 773-778; Li, S., et al., (2005) J Exp. Med 202, 617-624), neuropathic pain (White, F. A., et al., (2005) Proc. Natl. Acad. Sci. U.S.A 102, 14092-7, Jung et al. J Neurochem. 104, 254-63) and AIDS (Park, I. W., Wang, J. F., and Groopman, J. E. (2001) Blood 97, 352-358; Coll, B., et al., (2006) Cytokine 34, 51-55).

The mature form of MCP-1 is post-translationally modified by glutaminyl cyclase (QC) to possess an N-terminal pyroglutamyl (pGlu) residue (Proost, P. et al. (1996) J Leukocyte Biol. 59, 67-74).

Glutaminyl cyclase (QC, EC 2.3.2.5) catalyzes the intramolecular cyclization of N-terminal glutaminyl residues into pyroglutamic acid (5-oxo-proline, pGlu, pE) under liberation of ammonia and the intramolecular cyclization of N-terminal glutamyl residues into pyroglutamic acid under liberation of water (Fischer, W. H. and Spiess, J. (1987). Proc Natl Acad Sci USA 84: 3628-32, Golololov, M. Y., et al. (1994) Arch. Biochem. Biophys. 309, 300-7).

The N-terminal pGlu modification makes the protein resistant against N-terminal degradation by aminopeptidases, which is of prime importance, since chemotactic potency of MCP-1 is mediated by its N-terminus (Van Damme, J., et al., (1999) Chem Immunol 72, 42-56). Artificial elongation or degradation of the MCP-1 N-terminus (residues 1 to 9) leads to a dramatic decrease or loss of function, although MCP-1 still binds to its receptor (CCR2) (Proost, P., et al., (1998), J Immunol 160, 4034-4041; Zhang, Y. J., et al., 1994, J Biol. Chem 269, 15918-15924; Masure, S., et al., 1995, J Interferon Cytokine Res. 15, 955-963; Hemmerich, S., et al., (1999) Biochemistry 38, 13013-13025, Gong and Clark-Lewis (1995) J. Exp. Med. 181, 631-40).

Due to the prominent role of MCP-1 in a number of disease conditions, development and use of an anti-MCP-1 strategy required a potent tool for diagnostic, prognostic, and target modulation biomarker use.

As mentioned above, compelling evidence points to a role of MCP-1 in Alzheimer's disease (AD) (Xia, M. Q. and Hyman, B. T. (1999) J Neurovirol. 5, 32-41). The presence of MCP-1 in senile plaques and in reactive microglia, the residential macrophages of the CNS, has been observed in brains of patients suffering from AD (Ishizuka, K., et al., (1997) Psychiatry Clin. Neurosci. 51, 135-138. Stimulation of monocytes and microglia with Amyloid-β protein (Aβ) induces chemokine secretion in vitro (Meda, L., et al., (1996) J Immunol 157, 1213-1218; Szczepanik, A. M., et al., (2001) J Neuroimmunol. 113, 49-62) and intracerebroventricular infusion of Aβ (1-42) into murine hippocampus significantly increases MCP-1 in vivo. Moreover, Aβ deposits attract and activate microglial cells and force them to produce inflammatory mediators such as MCP-1, which in turn leads to a feed back to induce further chemotaxis, activation and tissue damage. At the site of Aβ deposition, activated microglia also phagocytose Aβ peptides leading to an amplified activation (Rogers, J. and Lue, L. F. (2001) Neurochem. Int. 39, 333-340).

Examination of chemokine expression in the 3xTg mouse model for AD revealed that neuronal inflammation precedes plaque formation and MCP-1 is upregulated by a factor of 11. Furthermore, the upregulation of MCP-1 seems to correlate with the occurrence of first intracellular Aβ deposits (Janelsins, M. C., et al., (2005) J Neuroinflammation. 2, 23). Cross-breeding of the Tg2575 mouse model for AD with a MCP-1 over-expressing mouse model has shown an increased microglia accumulation around Aβ deposits and that this accumulation was accompanied by an increased amount of diffuse plaques compared to single-transgenic Tg2576 littermates (Yamamoto, M., et al. (2005) Am. J Pathol. 166, 1475-1485).

MCP-1 levels are increased in the CSF of AD patients and patients showing mild cognitive impairment (MCI) (Galimberti, D., et al., (2006) Arch. Neurol. 63, 538-543). Furthermore, MCP-1 shows an increased level in the serum of patients with MCI and early AD (Clerici, F., et al., (2006) Neurobiol. Aging 27, 1763-1768).

Atherosclerotic lesions, which limit or obstruct coronary blood flow, are the major cause of ischemic heart disease related mortality, resulting in 500,000-600,000 deaths annually. Percutaneous transluminal coronary angioplasty (PTCA) to open the obstructed artery was performed in over 550,000 patients in the U.S. and 945,000+ patients worldwide in 1996 (Lemaitre et al., 1996). A major limitation of this technique is the problem of post-PTCA closure of the vessel, both immediately after PTCA (acute occlusion) and in the long term (restenosis): 30% of patients with subtotal lesions and 50% of patients with chronic total lesions will go on to restenosis after angioplasty. Additionally, restenosis is a significant problem in patients undergoing saphenous vein bypass graft. The mechanism of acute occlusion appears to involve several factors and may result from vascular recoil with resultant closure of the artery and/or deposition of blood platelets along the damaged length of the newly opened blood vessel followed by formation of a fibrin/red blood cell thrombus.

Restenosis after angioplasty is a more gradual process and involves initial formation of a subcritical thrombosis with release from adherent platelets of cell derived growth factors with subsequent proliferation of intimal smooth muscle cells and local infiltration of inflammatory cells contributing to vascular hyperplasia. It is important to note that multiple processes, among those thrombosis, cell proliferation, cell migration and inflammation each seem to contribute to the restenotic process.

In the U.S., a 30-50% restenosis rate translates to 120,000-200,000 U.S. patients at risk from restenosis. If only 80% of such patients elect repeated angioplasty (with the remaining 20% electing coronary artery bypass graft) and this is added to the cost of coronary artery bypass graft for the remaining 20%, the total cost for restenosis easily reaches into billions of dollars. Thus, successful prevention of restenosis could result not only in significant therapeutic benefit but also in significant health care savings.

Although it is not clear whether elevated MCP-1 expression is the cause or consequence of the above diseases, therapeutic benefit resulted from the application of MCP-1 neutralizing antibodies or MCP-1 receptor (CCR2) antagonists in a number of animal models.

In this context, it is important to note that deletion of amino acids 1-8 from the N-terminal region completely abolishes MCP-1 agonistic receptor activity, clearly indicating that the amino-terminal region is essential for receptor activation. (Gong, J.-H. and Clark-Lewis, I. (1995) J Exp. Med. 161 631-40, Van Damme, J., et al., (1999) Chem Immunol 72, 42-56).

Furthermore, any N-terminal MCP-1 truncation until residue 9 generates molecules with MCP-1 receptor antagonistic activity.

All existing assays for monitoring the MCP-1 level are not capable of distinguishing between N1pE MCP-1, N1Q MCP-1 and successively N-terminal truncated molecules. Therefore they do not reflect the degree of actual agonistics stimulation of the appropriate receptors (CCL2, CCL5) by full length MCP-1 in relation to the level of antagonistic effective N-terminal truncated or totally inactive MCP-1 molecules.

Within body fluids, only the N-terminal pyroglutamyl modified species of full length MCP-1 is detectable. The reason is the rapid N-terminal degradation of the N-terminal unmodified molecule by the resident ubiquitous aminopeptidase dipeptidyl aminopeptidase 4 (DP4, DPP4, DPPIV, CD26) and the aminopeptidase P (APP, X-prolyl aminopeptidase) liberating the N-terminal dipeptide Gln-Pro or the amino acid glutamine, respectively.

It consequently follows that the establishment of an assay displaying the real level of active, CCR2 receptor agonistic MCP-1 molecules, provides benefit to the improvement of diagnostic applications, monitoring therapeutic approaches and development of QC inhibitors in connection with diseases or disturbances in which MCP-1 might be involved.

Glutaminyl cyclase (QC, EC 2.3.2.5) catalyzes the intramolecular cyclization of N-terminal glutamine residues into pyroglutamic acid (pyroglutamate, pGlu, pE) liberating ammonia. A QC was first isolated by Messer from the latex of the tropical plant Carica papaya in 1963 (Messer, M. 1963 Nature 4874, 1299). 24 years later, a corresponding enzymatic activity was discovered in animal pituitary (Busby, W. H. J. et al. 1987 J Biol Chem 262, 8532-8536; Fischer, W. H. and Spiess, J. 1987 Proc Natl Acad Sci USA 84, 3628-3632). For the mammalian QC, the conversion of Gln into pGlu by QC could be shown for the precursors of TRH and GnRH (Busby, W. H. J. et al. 1987 J Biol Chem 262, 8532-8536; Fischer, W. H. and Spiess, J. 1987 Proc Natl Acad Sci USA 84, 3628-3632). In addition, initial localization experiments of QC revealed a co-localization with its putative products of catalysis in bovine pituitary, further improving the suggested function in peptide hormone synthesis (Bockers, T. M. et al. 1995 J Neuroendocrinol 7, 445-453). In contrast, the physiological function of the plant QC is less clear. In the case of the enzyme from C. papaya, a role in the plant defense against pathogenic microorganisms was suggested (El Moussaoui, A. et al. 2001 Cell Mol Life Sci 58, 556-570). Putative QCs from other plants were identified by sequence comparisons recently (Dahl, S. W. et al. 2000 Protein Expr Purif 20, 27-36). The physiological function of these enzymes, however, is still ambiguous.

The QCs known from plants and animals show a strict specificity for L-glutamine in the N-terminal position of the substrates and their kinetic behavior was found to obey the Michaelis-Menten equation (Pohl, T. et al. 1991 Proc Natl Acad Sci U S A 88, 10059-10063; Consalvo, A. P. et al. 1988 Anal Biochem 175, 131-138; Gololobov, M. Y. et al. 1996 Biol Chem Hoppe Seyler 377, 395-398). A comparison of the primary structures of the QCs from C. papaya and that of the highly conserved QC from mammals, however, did not reveal any sequence homology (Dahl, S. W. et al. 2000 Protein Expr Purif 20, 27-36). Whereas the plant QCs appear to belong to a new enzyme family (Dahl, S. W. et al. 2000 Protein Expr Purif 20, 27-36), the mammalian QCs were found to have a pronounced sequence homology to bacterial aminopeptidases (Bateman, R. C. et al. 2001 Biochemistry 40, 11246-11250), leading to the conclusion that the QCs from plants and animals have different evolutionary origins.

Recently, it was shown that recombinant human QC as well as QC-activity from brain extracts catalyze both the N-terminal glutaminyl as well as glutamate cyclization. Most striking is the finding that cyclase-catalyzed Glu1-conversion is favored around pH 6.0 while Gln1-conversion to pGlu-derivatives occurs with a pH-optimum of around 8.0. Since the formation of pGlu-Aβ-related peptides can be suppressed by inhibition of recombinant human QC and QC-activity from pig pituitary extracts, the enzyme QC is a target in drug development for the treatment of Alzheimer's disease.

Inhibitors of QC, which also could be useful as inhibitors of QC isoenzymes, are described in WO 2004/098625, WO 2004/098591, WO 2005/039548 and WO 2005/075436, which are incorporated herein in their entirety, especially with regard to the structure of the inhibitors, their use and their production. MCP-1 N1pE specific antibodies, useful for the detection and quantification of N-terminal pyroglutamate modified chemokine are described in International Patent Application No. PCT/EP2009/060757.

The inventors have now found that measurement of the proportion of N-terminal pyroglutamate modified MCP-1 in relation to the total concentration of MCP-1 within a sample provides an effective method for diagnosing or monitoring an inflammatory disease or an inflammatory associated disease.

### SUMMARY OF THE INVENTION

According to a first aspect of the invention, there is provided a method of diagnosing or monitoring an inflammatory disease or an inflammatory associated disease, which comprises determining the proportion of N-terminal pyroglutamate modified MCP-1 in relation to the total concentration of MCP-1 within a biological sample.

According to a second aspect of the invention, there is provided a method of determining the effectiveness of a glutaminyl cyclase (QC) inhibitor within a biological sample and as a surrogate marker for glutaminyl cyclase (QC) inhibition within a treatment by QC inhibitor application.

According to a third aspect of the invention, there is provided a method of determining the proportion of N-terminal pyroglutamate modified MCP-1 in relation to the total concentration of MCP-1 within a biological sample which comprises the following steps:
(a) determining a first concentration (cₐ) of N-terminal pyroglutamate modified MCP-1 in a biological sample;
(b) determining a second concentration (c_{d}) of total MCP-1 in said biological sample; and
(c) determining the ratio of cₐ/c_{d}, wherein the value of the first concentration (cₐ) is divided by the value of the second concentration (c_{d}).

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1: Sequence alignment of mature MCP-1 (CCL2) proteins from different species. Alignment was performed using CLUSTAL W (1.83) multiple sequence alignment algorithm provided at http://www.ch.embnet.org/software/ClustalW.html. Sequences are: human: human MCP-1 SEQ ID NO: 1, chimp: chimpanzee MCP-1 SEQ ID NO: 2, oran: Sumatran orang-utan MCP-1 SEQ ID NO: 3, macac: Macaca fascicularis (Crab eating macaque) MCP-1 SEQ ID NO: 4, doc: Canis familiaris MCP-1 SEQ ID NO: 5, pig: Sus scrofa MCP-1, SEQ ID NO: 6, cow: Bos taurus MCP-1, SEQ ID NO: 7, horse: Equus caballus MCP-1, SEQ ID NO: 8, mouse: Mus musculus MCP-1, SEQ ID NO: 9, rat: Rattus norvegicus MCP-1, SEQ ID NO: 10.

FIG. 2: Alignment of the four human MCP's. Alignment was performed using CLUSTAL W (1.83) multiple sequence alignment algorithm provided at http://www.ch.embnet.org/software/ClustalW.html. Sequences are: MCP-1: human MCP-1 (CCL2, SCYA2, MCAF, SMC-CF, GDCF-2, HC11 SEQ ID NO: 1, MCP-2: human MCP-2 (CCL8, SCYA8, HC14), SEQ ID NO: 11; MCP-3: human MCP-3 (CCL7, SCYA7, NC28, FIC, MARC), SEQ ID NO: 12; MCP-4: human MCP-4 (CCL13, SCYA13, NCC-1, CKb10), SEQ ID NO: 13. The N-terminal residues typed in bold letters indicate the signal sequence which is removed in mature chemokines The arrow marks the emerging N-terminal glutamine residues forming the pyroglutamyl derivative catalyzed by glutaminyl cyclases.

FIG. 3: shows the incubation of MCP-1(1-76) bearing an N-terminal glutaminyl residue with recombinant human DP4 for 24 h. The DP4 cleavage products were analyzed after 0 min, 15 min, 30 min, 1 h, 4 h and 24 h using Maldi-TOF mass spectrometry.

FIG. 4: shows the incubation of MCP-1(1-76) bearing an N-terminal Pyroglutamyl (5-oxo-L-Prolyl) residue with recombinant human DP4 for 24 h. For cyclization of N-terminal glutamine into pyroglutamate MCP-1 was incubated with recombinant human QC 3 h prior to assay start. The cleavage was analyzed after 0 min, 15 min, 30 min, 1 h, 4 h and 24 h using Maldi-TOF mass spectrometry.

FIG. 5: illustrates cleavage of human MCP-1(1-76) bearing an N-terminal glutaminyl residue by recombinant human Aminopeptidase P for 24 h. The APP cleavage products were analyzed after 0 min, 15 min, 30 min, 1 h, 2 h, 4 h and 24 h using Maldi-TOF mass spectrometry.

FIG. 6: illustrates cleavage of human MCP-1(1-76) bearing an N-terminal pyroglutamyl (5-oxo-L-Prolyl) residue by recombinant human Aminopeptidase P for 24 h. The pyroglutamate formation at the N-Terminus was accomplished by incubation of MCP-1 with recombinant human QC for 3 h prior to the assay. The APP cleavage was analyzed after 0 min, 15 min, 30 min, 1 h, 2 h, 4 h and 24 h using Maldi-TOF mass spectrometry.

FIG. 7: shows the degradation of human MCP-1(1-76) carrying an N-terminal glutaminyl residue (A) or pyroglutamyl (5-oxo-L-Prolyl) residue (B) in human serum for 7 and 24 h, respectively. For cyclization of the N-terminal glutamine residue into pyroglutamate, MCP-1 was incubated with recombinant human QC for 3 h prior to assay start. In addition, Gln¹-MCP-1 was incubated in human serum in the presence of 9.6 μM DP4 Inhibitor Isoleucyl-Thiazolidide (P32/98) for 24 h (C). The cleavage products were analyzed after 0 min, 10 min, 30 min, 1 h, 2 h, 3 h 5 h and 7 h for Gln¹-MCP-1, 0 min, 30 min, 1 h, 2 h, 3 h 5 h, 7 h and 24 h for pGlu¹-MCP-1 and 0 min, 1 h, 2 h, 3 h, 5 h, 7 h and 24 h for Gln¹-MCP-1 in combination with Isoleucyl-Thiazolidide using Maldi-TOF mass spectrometry.

FIG. 8: shows the degradation of human MCP-2(1-76) bearing an N-terminal glutaminyl (A) or pyroglutamyl (5-oxo-L-Prolyl) residue (B) by recombinant human DP4 for 24 h. For cyclization of N-terminal glutamine into pyroglutamate, MCP-2 was incubated with recombinant human QC for 3 h prior to assay start. The DP4 cleavage products were analyzed using Maldi-TOF mass spectrometry after 0 min, 15 min, 30 min, 1 h, 2 h, 4 h and 24 h.

FIG. 9: shows the degradation of human MCP-3(1-76) carrying an N-terminal glutaminyl (A) or pyroglutamyl (5-oxo-L-Prolyl) residue (B) by recombinant human DP4 for 24 h. For cyclization of N-terminal glutamine into pyroglutamate, MCP-3 was incubated with recombinant human QC for 3 h prior to assay start. The DP4 cleavage products were analyzed using Maldi-TOF mass spectrometry after 0 min, 15 min, 30 min, 1 h, 2 h, 4 h and 24 h.

FIG. 10: illustrates the cleavage of human MCP-4(1-75) bearing an N-terminal glutaminyl (A) or pyroglutamyl (5-oxo-L-Prolyl) residue (B) by recombinant human DP4 for 4 hours. For cyclization of N-terminal glutamine into pyroglutamate, MCP-4 was incubated with recombinant human QC for 3 h prior to assay start. The DP4 cleavage products were analyzed using Maldi-TOF mass spectrometry after 0 min, 15 min, 30 min, 1 h, 2 h, and 4 h.

FIG. 11: shows the chemotactic potency of human N-terminal MCP-1 starting with N-terminal glutamine(Gln1-MCP-1) in comparison to human MCP-1 with N-terminal pyroglutamic acid(pGlu1-MCP-1) (A), of human N-terminal MCP-2 starting with N-terminal glutamine(Gln1-MCP-2) in comparison to human MCP-2 with N-terminal pyroglutamic acid(pGlu1-MCP-2) (B), of human N-terminal MCP-3 starting with N-terminal glutamine(Gln1-MCP-3) in comparison to human MCP-3 with N-terminal pyroglutamic acid(pGlu1-MCP-3) (C) and of human N-terminal MCP-4 starting with N-terminal glutamine(Gln1-MCP-4) in comparison to human MCP-4 with N-terminal pyroglutamic acid(pGlu1-MCP-4) (D), towards human THP-1 monocytes.

FIG. 12: shows the analysis of chemotactic potency of human MCP-1 towards human THP-1 monocytes, which was incubated with human recombinant DP4 in the presence (Gln¹-MCP-1+QC+DP4) and absence (Gln¹-MCP-1+DP4) of QC-mediated pGlu formation. In addition, the influence of the QC-inhibitor 1-(3-(1H-imidazol-1-yl)propyl)-3-(3,4-dimethoxyphenyl)thiourea hydrochloride (QCI) (10 μM) on the formation of the N-terminal pGlu-residue, followed by subsequent DP4 cleavage (Gln¹-MCP-1+QC+QCI+DP4) is shown.

FIG. 13: shows the chemotactic potency of full-length human MCP-1 (A), MCP-3 (B), MCP-2 (C) and MCP-4 (D) starting with an N-terminal glutamine in comparison to their respective DP4 cleavage products towards human THP-1 monocytes.

FIG. 14: Standard curves for the determination of A: total hMCP-1 and B: hMCP-1 N1pE concentrations on the basis of measured absorption values at 450 nm/540 nm. For both standard curves, recombinant hMCP-1 N1pE produced in E. coli was used. The standard curve was calculated from measured absorption data by a 4-Parameter-Logistic-Fit: y=(A2+(A1−A2)/(1+(x/x0)̂p).

FIG. 15: Comparison of the detection of hMCP-1 N1pE and hMCP-1 in the total hMCP-1 sandwich ELISA.

FIG. 16: Time dependent expression of total hMCP-1 and hMCP-1 N1pE by NHDF after stimulation with OSM and IL1β.

FIG. 17: Time dependent expression of A: total hMCP-1 and B: hMCP-1 N1pE by NHDF after stimulation with OSM+IL1β and application of QCI. C: Ratio of hMCP-1 N1pE/hMCP-1.

FIG. 18: A: Expression of total hMCP-1 and hMCP-1 N1pE by A549 cells after stimulation with TNFα+IL1β and application of different QCI concentrations. B: Ratio of hMCP-1 N1pE/hMCP-1.

FIG. 19: Standard curves for the determination of mouse MCP-1; A: total mMCP1 and B: mMCP-1 N1pE concentrations on the basis of measured absorption values at 450 nm/540 nm. For both standard curves, recombinant mouse MCP-1 N1pE produced in E. coli was used. The standard curve was calculated from measured absorption data by a 4-Parameter-Logistic-Fit: y=(A2+(A1−A2)/(1+(x/xₒ)̂p).

FIG. 20: Comparison of the detection of mMCP-1 N1pE and mMCP-1 in the total mMCP-1 sandwich ELISA.

FIG. 21: A: Expression of total mMCP-1 and mMCP-1 N1pE by RAW 264.7 cells after stimulation with 10 ng/ml LPS and application of different QCI concentrations. B: Ratio of mMCP-1 N1pE/mMCP-1.

FIG. 22: Western Blot signals of A: mMCP-1 N1pE and B: total mMCP-1 in cell culture supernatant of RAW 264.7 cells after stimulation with 10 ng/ml LPS and application of different QCI. C: Concentrations of mMCP-1 N1pE determined by ELISA.

FIG. 23: A: Amounts of total mMCP-1 and mMCP-1 N1pE in mouse peritoneal lavage fluid after stimulation with thioglycollate and application of different QCI concentrations. B: FACS analysis: Fluorescence Events after double monocyte staining in peritoneal lavage fluid with anti 7/4 and Ly6G antibodies.

FIG. 24: Standard curves for the determination of mouse MCP-1 N1pE; A: mMCP-1 N1pE detection by MCP-1 N1pE antibody clone 348/2C9 and B: mMCP-1 N1pE detection by biotinylated MCP-1 N1pE antibody clone 348/2C9. For both standard curves, recombinant mouse MCP-1 N1pE produced in E. coli was used. The standard curve was calculated from measured absorption data by a 4-Parameter-Logistic-Fit: y=(A2+(A1−A2)/(1+(x/X₀)̂p).

FIG. 25: Isothermal titration calorimetry measurement of anti-MCP-1 N1pE antibodies (A: MCP-1 N1pE antibody 348/2C9 and B: biotinylated MCP-1 N1pE antibody 348/2C9) to the antigen hMCP-1(1-38).

FIG. 26: Measurement of human MCP-1 and human MCP-1 N1pE in CSF and serum samples derived from 10 healthy volunteers by ELISA.

### BRIEF DESCRIPTION OF SEQUENCE LISTING

TABLE 1
Sequence listing

| Sequence No. | Description |
|---|---|
| 1 | human MCP-1 |
| 2 | Pan troglodytes (chimpanzee) MCP-1 |
| 3 | Pongo abelii (Sumatran orang-utan) MCP-1 |
| 4 | Macaca fascicularis (Crab eating macaque) MCP-1 |
| 5 | Canis familiaris MCP-1 |
| 6 | Sus scrofa MCP-1 |
| 7 | Bos taurus MCP-1 |
| 8 | Equus caballus MCP-1 |
| 9 | Mus musculus MCP-1 |
| 10 | Rattus norvegicus MCP-1 |
| 11 | human MCP-2 |
| 12 | human MCP-3 |
| 13 | human MCP-4 |

### DEFINITIONS

“Dectection antibody” in the sense of the present application is intended to encompass those antibodies which bind to MCP-1 or the N-terminal pyroglutamate modified MCP-1 peptide.

Suitably the dectection antibodies bind to MCP-1 or the N-terminal pyroglutamate modified MCP-1 peptide with a high affinity. In the context of the present invention, high affinity means an affinity with a K_{D} value of 10⁻⁷M or better, such as a K_{D} value of 10⁻⁸M or better or even more particularly, a K_{D} value of 10⁻⁹M to 10⁻¹²M.

The term “antibody” is used in the broadest sense and specifically covers intact monoclonal antibodies, polyclonal antibodies, multispecific antibodies (e.g. bispecific antibodies) formed from at least two intact antibodies, and antibody fragments as long as they exhibit the desired biological activity. The antibody may be an IgM, IgG (e.g. IgG1, IgG2, IgG3 or IgG4), IgD, IgA or IgE, for example. Suitably however, the antibody is not an IgM antibody. The “desired biological activity” is binding to MCP-1 or the N-terminal pyroglutamate modified MCP-1 peptide.

“Antibody fragments” comprise a portion of an intact antibody, generally the antigen binding or variable region of the intact antibody. Examples of antibody fragments include Fab, Fab′, F(ab′)2, and Fv fragments: diabodies; single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to “polyclonal antibody” preparations which typically include different antibodies directed against different determinants (epitopes), each monoclonal antibody is directed against a single determinant on the antigen. In addition to their specificity, the monoclonal antibodies can frequently be advantageous in that they are synthesized by the hybridoma culture, uncontaminated by other immunoglobulins. The “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Köhler et al., Nature, 256:495 (1975), or may be made by generally well known recombinant DNA methods. The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain a minimal sequence derived from a non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences.

These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986), Reichmann et al, Nature. 332:323-329 (1988): and Presta, Curr. Op. Struct. Biel., 2:593-596 (1992). The humanized antibody includes a Primatized™ antibody wherein the antigen-binding region of the antibody is derived from an antibody produced by immunizing macaque monkeys with the antigen of interest or a “camelized” antibody.

“Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{D}) in the same polypeptide chain (V_{H}-V_{D}). By using a linker that is too short to allow pairing between the two domains on the same chain, the domains are forced to pair with the complementary domains of another chain and create two antigen-binding sites. Diabodies are described more fully in Hollinger et al., Proc. Natl. Acad. Sol. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. In suitable embodiments, the antibody will be purified (1) to greater than 95% by weight of antibody as determined by the Lowry method, and most particularly more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, suitably, silver stain. Isolated antibody includes the antibody in situ within recombinant cells since at least one component of the antibody's natural environment will not be present. Ordinarily, however, isolated antibody will be prepared by at least one purification step.

As used herein, the expressions “cell”, “cell line,” and “cell culture” are used interchangeably and all such designations include progeny. Thus, the words “transformants” and “transformed cells” include the primary subject cell and culture derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, this will be clear from the context.

The terms “polypeptide”, “peptide”, and “protein”, as used herein, are interchangeable and are defined to mean a biomolecule composed of amino acids linked by a peptide bond.

“Homology” between two sequences is determined by sequence identity. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs such as the Bestfit program (Wisconsin Sequence Analysis Package, Version 8 for Unix, Genetics Computer Group, University Research Park, 575 Science Drive Madison, Wis. 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has, for example, 95% identity with a reference sequence of the present invention, the parameters are preferably adjusted so that the percentage of identity is calculated over the entire length of the reference sequence and homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W. R. Pearson (1990), Methods in Enzymology 183, 63-98, appended examples and http://workbench.sdsc.edu/). For this purpose, the “default” parameter settings may be used.

As used herein, a “conservative change” refers to alterations that are substantially conformationally or antigenically neutral, producing minimal changes in the tertiary structure of the mutant polypeptides, or producing minimal changes in the antigenic determinants of the mutant polypeptides, respectively, as compared to the native protein. When referring to the antibodies and antibody fragments of the invention, a conservative change means an amino acid substitution that does not render the antibody incapable of binding to the subject receptor. One of ordinary skill in the art will be able to predict which amino acid substitutions can be made while maintaining a high probability of being conformationally and antigenically neutral. Such guidance is provided, for example in Berzofsky, (1985) Science 229:932-940 and Bowie et al. (1990) Science 247: 1306-1310. Factors to be considered that affect the probability of maintaining conformational and antigenic neutrality include, but are not limited to: (a) substitution of hydrophobic amino acids is less likely to affect antigenicity because hydrophobic residues are more likely to be located in a protein's interior; (b) substitution of physicochemically similar, amino acids is less likely to affect conformation because the substituted amino acid structurally mimics the native amino acid; and (c) alteration of evolutionarily conserved sequences is likely to adversely affect conformation as such conservation suggests that the amino acid sequences may have functional importance. One of ordinary skill in the art will be able to assess alterations in protein conformation using well-known assays, such as, but not limited to microcomplement fixation methods (see, e.g. Wasserman et al. (1961) J. Immunol. 87:290-295; Levine et al. (1967) Meth. Enzymol. 11:928-936) and through binding studies using conformation-dependent monoclonal antibodies (see, e.g. Lewis et al. (1983) Biochem. 22:948-954).

The terms “a”, “an” and “the” as used herein are defined to mean one or more and include the plural unless the context is inappropriate.

### Inflammatory Diseases

The terms “inflammatory disease” and “inflammatory associated disease” as used herein comprises:
(a) neurodegenerative diseases, e.g. mild cognitive impairment (MCI), Alzheimer's disease, neurodegeneration in Down Syndrome, Familial British Dementia, Familial Danish Dementia, multiple sclerosis;
(b) chronic and acute inflammations, e.g. rheumatoid arthritis, atherosclerosis, restenosis, pancreatitis;
(c) fibrosis, e.g. lung fibrosis, liver fibrosis, renal fibrosis;
(d) cancer, e.g. cancer/hemangioendothelioma proliferation, gastric carcinomas;
(e) metabolic diseases, e.g. hypertension;
(f) other inflammatory diseases, e.g. neuropathic pain, graft rejection/graft failure/graft vasculopathy, HIV infections/AIDS, gestosis, tuberous sclerosis; and
(g) pathologies associated with hyperinsulinemia and obesity.

### QC

The term “QC” as used herein comprises glutaminyl cyclase (QC) and QC-like enzymes. QC and QC-like enzymes have identical or similar enzymatic activity, further defined as QC activity. In this regard, QC-like enzymes can fundamentally differ in their molecular structure from QC.

The term “QC activity” as used herein is defined as intramolecular cyclization of N-terminal glutaminyl residues into pyroglutamic acid (pGlu*) or of N-terminal L-homoglutaminyl or L-beta-homoglutaminyl to a cyclic pyro-homoglutamine derivative under liberation of ammonia. See schemes 1 and 2 in this regard.

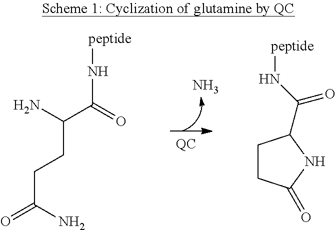

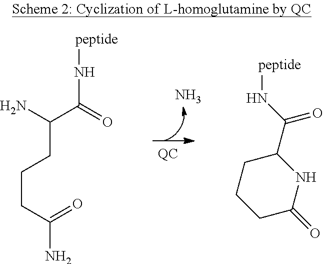

The term “EC” as used herein comprises the side activity of QC and QC-like enzymes as glutamate cyclase (EC), further defined as EC activity.

The term “EC activity” as used herein is defined as intramolecular cyclization of N-terminal glutamyl residues into pyroglutamic acid (pGlu*) by QC. See scheme 3 in this regard.

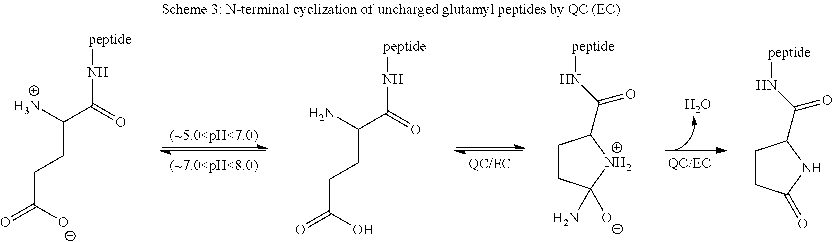

The term “QC-inhibitor” and “glutaminyl cyclase inhibitor” is generally known to a person skilled in the art and means enzyme inhibitors as generally defined above, which inhibit the catalytic activity of glutaminyl cyclase (QC) or its glutamyl cyclase (EC) activity.

### Potency of QC Inhibition

In light of the correlation with QC inhibition, in preferred embodiments, the subject method and medical use utilize an agent with a Kᵢ for QC inhibition of 10 μM or less, more preferably of 1 μM or less, even more preferably of 0.1 μM or less or 0.01 μM or less, or most preferably 0.001 μM or less. Indeed, inhibitors with Kᵢ values in the lower micromolar, preferably the nanomolar and even more preferably the picomolar range are contemplated. Thus, while the active agents are described herein, for convenience, as “QC inhibitors”, it will be understood that such nomenclature is not intended to limit the subject matter of the invention in any way.

Examples of glutaminyl cyclase inhibitors are described in WO 2005/075436, in particular examples 1-141 as shown on pp. 31-40. The synthesis of examples 1-141 is shown on pp. 40-48 of WO 2005/075436. The disclosure of WO 2005/075436 regarding examples 1-141, their synthesis and their use as glutaminyl cyclase inhibitors is incorporated herein by reference.

Further examples of inhibitors of glutaminyl cyclase are described in WO 2008/055945, in particular examples 1-473 as shown on pp. 46-155. The synthesis of examples 1-473 is shown on pp. 156-192 of WO 2008/055945. The disclosure of WO 2008/055945 regarding examples 1-473, their synthesis and their use as glutaminyl cyclase inhibitors is incorporated herein by reference.

Further examples of inhibitors of glutaminyl cyclase are described in WO 2008/055947, in particular examples 1-345 as shown on pp. 53-118. The synthesis of examples 1-345 is shown on pp. 119-133 of WO 2008/055947. The disclosure of WO 2008/055947 regarding examples 1-345, their synthesis and their use as glutaminyl cyclase inhibitors is incorporated herein by reference.

Further examples of inhibitors of glutaminyl cyclase are described in WO 2008/055950, in particular examples 1-212 as shown on pp. 57-120. The synthesis of examples 1-212 is shown on pp. 121-128 of WO 2008/055950. The disclosure of WO 2008/055950 regarding examples 1-212, their synthesis and their use as glutaminyl cyclase inhibitors is incorporated herein by reference.

Further examples of inhibitors of glutaminyl cyclase are described in WO2008/065141, in particular examples 1-25 as shown on pp. 56-59. The synthesis of examples 1-25 is shown on pp. 60-67 of WO2008/065141. The disclosure of WO2008/065141 regarding examples 1-25, their synthesis and their use as glutaminyl cyclase inhibitors is incorporated herein by reference.

Further examples of inhibitors of glutaminyl cyclase are described in WO 2008/110523, in particular examples 1-27 as shown on pp. 55-59. The synthesis of examples 1-27 is shown on pp. 59-71 of WO 2008/110523. The disclosure of WO 2008/110523 regarding examples 1-27, their synthesis and their use as glutaminyl cyclase inhibitors is incorporated herein by reference.

Further examples of inhibitors of glutaminyl cyclase are described in WO 2008/128981, in particular examples 1-18 as shown on pp. 62-65. The synthesis of examples 1-18 is shown on pp. 65-74 of WO 2008/128981. The disclosure of WO 2008/128981 regarding examples 1-18, their synthesis and their use as glutaminyl cyclase inhibitors is incorporated herein by reference.

Further examples of inhibitors of glutaminyl cyclase are described in WO 2008/128982, in particular examples 1-44 as shown on pp. 61-67. The synthesis of examples 1-44 is shown on pp. 68-83 of WO 2008/128982. The disclosure of WO 2008/128982 regarding examples 1-44, their synthesis and their use as glutaminyl cyclase inhibitors is incorporated herein by reference.

Further examples of inhibitors of glutaminyl cyclase are described in WO 2008/128983, in particular examples 1-30 as shown on pp. 64-68. The synthesis of examples 1-30 is shown on pp. 68-80 of WO 2008/128983. The disclosure of WO 2008/128983 regarding examples 1-30, their synthesis and their use as glutaminyl cyclase inhibitors is incorporated herein by reference.

Further examples of inhibitors of glutaminyl cyclase are described in WO 2008/128984, in particular examples 1-36 as shown on pp. 63-69. The synthesis of examples 1-36 is shown on pp. 69-81 of WO 2008/128984. The disclosure of WO 2008/128984 regarding examples 1-36, their synthesis and their use as glutaminyl cyclase inhibitors is incorporated herein by reference.

Further examples of inhibitors of glutaminyl cyclase are described in WO 2008/128985, in particular examples 1-71 as shown on pp. 66-76. The synthesis of examples 1-71 is shown on pp. 76-98 of WO 2008/128985. The disclosure of WO 2008/128985 regarding examples 1-71, their synthesis and their use as glutaminyl cyclase inhibitors is incorporated herein by reference.

Further examples of inhibitors of glutaminyl cyclase are described in WO 2008/128986, in particular examples 1-7 as shown on pp. 65-66. The synthesis of examples 1-7 is shown on pp. 66-73 of WO 2008/128986. The disclosure of WO 2008/128986 regarding examples 1-7, their synthesis and their use as glutaminyl cyclase inhibitors is incorporated herein by reference.

### Western Blot

Western blot analysis, also known as immuno- or protein blotting, is used to detect specific proteins from a heterogeneous sample. The protocol was first developed by Harry Towbin, et al. (1979) using a nitrocellulose membrane.

The method is composed of four main steps,

First: Sodium dodecyl sulfate polyacrylamide gel electrophoresis (SDS PAGE) of the protein sample.

Second: Electrophoretic transfer (blotting) to a membrane. For transfer, later researchers introduced different membranes, most notably the PVDF nylon-like membrane, which enabled sequencing of isolated proteins.

Third: Labeling of target protein(s) with specific primary and secondary antibodies. Unspecific antibody binding is prevented by incubation of the membrane in blocking solution for 1 hour at room temperature or at 4° C. overnight with shaking. The blocking solution is normally composed of 5% non-fat milk in TBS-T, although some antibodies require BSA in place of milk. This is normally clear in the manufacturers instructions for the antibody for testing. Incubate primary antibody overnight or at room temperature for 2 hours. Incubate membrane with an appropriate secondary antibody (e.g. peroxidase conjugated) for 1 hour at room temperature.

Fourth: Detection and imaging of target protein(s). There are numerous chemiluminescence reagents available commercially (Amersham, Pierce, Invitrogen) with each manufacturer selling a range of sensitivities of detection levels. These typically take the form of two solutions which are combined and then incubated immediately on the membrane for 1-5 minutes. Expose membrane to X-ray film for 1 minute to 1 hour, depending on protein signal and chemiluminescence method. The secondary antibody can be conjugated with other enzymes (alkaline phosphatase) and therefore visualized with the corresponding substrates using alternative protocols.

### ELISA

ELISA (enzyme-linked immunosorbent assay) is one of the most widely used quantitative tools for sensitive and reproducible analyte assays. The technique combines the high specificity of an antibody-antigen interaction with an enzyme-linked signal detection system.

### Indirect ELISA

In an indirect ELISA, the antigen is immobilized to a surface, the detection is provided by the specific antibody enzyme conjugate complex with subsequent staining.

In the first step, the investigated antigen is immobilized to a surface in a number of known concentrations to achieve a standard curve. At the same conditions, the sample with the unknown amount of antigen is immobilized. The antigen specific antibody recognises the antigen. If this antibody is linked to an enzyme (or a second enzyme-conjugated antibody recognises the primary antibody), the signal of appropriate enzymatic reaction using a chromogenic or fluorogenic substrate is in correlation to the amount of the antigen and can be computed by the means of the standard curve.

### “Sandwich” ELISA

A “sandwich” ELISA is a technique in which an antigen is sandwiched between two different antibodies. The principle by which this ELISA technique operates is as follows:
Immobilization of capture antibody on a suitable substrate
Binding of antigen to immobilized antibody
Binding of second antibody, linked to an enzyme, to bound antigen (formation of immune complex)
Detection of immune complex using appropriate enzyme substrate

### Competitive ELISA

The steps for this ELISA are:
Unlabeled antibody is incubated in the presence of its antigen.
These antibody/antigen complexes are then added to an antigen coated well.
The plate is washed, so that unbound antibody is removed. (The more antigen in the sample, the less antibody will be able to bind to the antigen in the well, hence “competition.”)
The secondary antibody, specific to the primary antibody is added. This second antibody is coupled to the enzyme.
A substrate is added, and remaining enzymes elicit a chromogenic or fluorescent signal.

For competitive ELISA, the higher the original antigen concentration, the weaker the eventual signal. The major advantage of a competitive ELISA is the ability to use crude or impure samples and still selectively bind any antigen that may be present.

Some competitive ELISA kits include enzyme-linked antigen rather than enzyme-linked antibody. The labeled antigen competes for primary antibody binding sites with the sample antigen (unlabeled). The more antigen in the sample, the less labeled antigen is retained in the well and the weaker the signal).

### Reverse ELISA

The technique uses a solid phase made up of an immunosorbent polystyrene rod with 4-12 protruding ogives. The entire device is immersed in a test tube containing the collected sample and the following steps (washing, incubation in conjugate and incubation in chromogenous) are carried out by dipping the ogives in microwells of standard microplates pre-filled with reagents.

### Enzyme-Linked Immunosorbent Spot Assay

The Enzyme-linked immunosorbent spot (ELISPOT) assay is a common method for monitoring immune responses in humans and animals. It allows visualization of the secretory product of individual activated or responding cells. A capture antibody is coated aseptically onto a PVDF-backed microplate. The plate is blocked, usually with a serum protein that is non-reactive with any of the antibodies in the assay. After this, cells of interest are plated out at varying densities, along with antigen or mitogen, and then placed in a humidified 37° C. CO₂ incubator for a specified period of time.

Cytokine (or other cell product of interest) secreted by activated cells is captured locally by the coated antibody on the high surface area PVDF membrane. After washing the wells to remove cells, debris, and media components, a biotinylated polyclonal antibody specific for the chosen analyte is added to the wells. This antibody is reactive with a distinct epitope of the target cytokine and thus is employed to detect the captured cytokine. Following a wash to remove any unbound biotinylated antibody, the detected cytokine is then visualized using an avidin-HRP, and a precipitating substrate (e.g., AEC, BCIP/NBT). The colored end product (a spot, usually a blackish blue) typically represents an individual cytokine-producing cell. The spots can be counted manually (e.g., with a dissecting microscope) or using an automated reader to capture the microwell images and to analyze spot number and size.

The FluoroSpot assay is a modification of the ELISPOT assay and is based on using multiple fluoroscent anticytokines which makes it possible to spot two cytokines in the same assay.

### Flow Cytometry

### Intracellular Flow Cytometry (ICFC)

In contrast to detection of secreted cytokines by ELISA, for detection of intracellular cytokines, it is necessary to block secretion of cytokines with protein transport inhibitors, such as Monensin or Brefeldin A, during the last few hours of the stimulation. It is advised that the investigators evaluate the use and efficacy of different protein transport inhibitors in their specific assay system.

A modification of the basic immunofluorescent staining and flow cytometric analysis protocol can be used for the simultaneous analysis of surface molecules and intracellular antigens at the single-cell level. In this protocol, cells are first activated in vitro, stained for surface antigens as in the surface antigen protocol, then fixed with paraformaldehyde to stabilize the cell membrane and permeabilized with the detergent saponin to allow anti-cytokine antibodies to stain intracellularly. In vitro stimulation of cells is usually required for detection of cytokines by flow cytometry since cytokine levels are typically too low in resting cells. Stimulation of cells with the appropriate reagent will depend on the cell type and the experimental conditions.

### Flow Cytometry Using Multiplex Assay Technology

Luminex xMAP Technology

The xMAP technology uses 5.6 micron polystyrene microspheres which are internally dyed with red and infrared fluorophores. Using different amounts of the two dyes for different batches of microspheres, up to 100 different microsphere sets can be created. Each bead is unique with a spectral signature determined by a red and infrared dye mixture. The bead is filled with a specific known ratio of the two dyes. As each microsphere carries a unique signature, the xMAP detection system can identify to which set it belongs. Therefore, multiplexing up to 100 tests in a single reaction volume is possible.

### Luminex Assay

The Luminex System is a flexible analyzer based on the principles of flow cytometry. The system enables to multiplex (simultaneously measure) up to 100 analytes in a single microplate well, using very small sample volumes. Analysis of multiplexed solutions of up to 40 different analytes in a single well are possible. The system delivers bioassays which include gene expression, transcription factor profiling, cytokine profiling etc.

### Bio-Plex Assay

The Bio-Plex cytokine assay employs a liquid suspension array for quantification of cytokines in tissue culture supernatants or serum. Using this 96-well microtiter plate-formatted assay, it is possible to profile the level of multiple cytokines in a single well. The principle of the Bio-Plex cytokine assay is similar to a capture sandwich immunoassay. An antibody directed against each desired cytokine is covalently coupled to a different color-coded polystyrene bead. The conjugated beads are allowed to react with a sample containing a known (standard) or unknown amount of cytokines. After unbound cytokines are removed, biotinylated detection antibodies directed against a different epitope on each cytokine are added to the reaction. The result is the formation of a sandwich of antibodies around each cytokine. The complexes are detected by the addition of streptavidin-phycoerythrin (streptavidin-PE), which has fluorescence characteristics distinct from the beads. A specialized microtiter plate reader, which allows for analysis of multiplexed bead-capture immunoassays in a single microtiter well, carries out quantification. By reading beads individually in the mixture, the system can detect each cytokine separately. The Bio-Plex software automatically calculates the concentration of cytokines from standard curves derived from a mixture of cytokine standards of a known amount.

### Immunohistochemistry (IHC)

Immunohistochemistry or IHC refers to the process of localizing antigens (eg. proteins) in cells of a tissue section. IHC is also widely used in basic research to understand the distribution and localization of biomarkers and differentially expressed proteins in different parts of a biological tissue. Visualising an antibody-antigen interaction can be accomplished in a number of ways. In the most common instance, an antibody is conjugated to an enzyme, such as peroxidase, that can catalyse a colour-producing reaction. Alternatively, the antibody can also be tagged to a fluorophore, such as fluorescein, rhodamine, DyLight Fluor or Alexa Fluor.

Antibodies can be classified as primary or secondary reagents. Primary antibodies are raised against an antigen of interest and are typically unconjugated (unlabelled), while secondary antibodies are raised against primary antibodies. Hence, secondary antibodies recognize immunoglobulins of a particular species and are conjugated to either biotin or a reporter enzyme such as alkaline phosphatase or horseradish peroxidase (HRP). Some secondary antibodies are conjugated to fluorescent agents. In the procedure, depending on the purpose and the thickness of the experimental sample, either thin (about 4-40 μm) slices are taken from the tissue of interest, or if the tissue is not very thick and is penetrable it is used whole. The slicing is usually accomplished through the use of a microtome, and slices are mounted on slides. “Free-floating IHC” uses slices that are not mounted, these slices are normally produced using a vibrating microtome.

The direct method is a one-step staining method, and involves a labeled antibody reacting directly with the antigen in tissue sections.

The indirect method involves an unlabeled primary antibody (first layer) which reacts with tissue antigen, and a labeled secondary antibody (second layer) which reacts with the primary antibody.

### Immunoprecipitation (IP)

Immunoprecipitation involves using an antibody that is specific for a known protein to isolate that particular protein out of a solution containing many different proteins. These solutions will often be in the form of a crude lysate of a plant or animal tissue. Other sample types could be bodily fluids or other samples of biological origin.

### DETAILED DESCRIPTION OF THE INVENTION

According to a first aspect of the invention, there is provided a method of diagnosing or monitoring an inflammatory disease or an inflammatory associated disease, which comprises determining the proportion of N-terminal pyroglutamate modified MCP-1 in relation to the total concentration of MCP-1 within a biological sample.

According to a second aspect of the invention, there is provided a method of determining the effectiveness of a glutaminyl cyclase (QC) inhibitor within a biological sample and as a surrogate marker for glutaminyl cyclase (QC) inhibition within a treatment by QC inhibitor application.

The data presented herein surprisingly demonstrate that a decreased ratio of N-terminal pyroglutamate modified MCP-1(MCP-1 N1pE): total concentration of MCP-1 consequently resulted in a decreased number of infiltrating monocytes to the peritoneum. Such a recruitment of monocytes is a general feature of several inflammatory disorders. This data therefore proves the applicability of the MCP-1 N1pE/MCP-1 ratio as a biomarker for inflammatory diseases or inflammatory associated diseases by monitoring the monocyte recruitment capacity of MCP-1.

References herein to “MCP-1” apply to an MCP-1 peptide having greater than 50% sequence identity (such as any one of 55, 60, 65, 70, 75, 80, 85, 90, 95, 96, 97, 98, 99 or 100%) to any of SEQ ID NOs 1-10. In one embodiment, the MCP-1 is MCP-1 (1-76). In a further embodiment, the MCP-1 is human or mouse MCP-1. In a yet further embodiment, the MCP-1 is human MCP-1.

References to “N-terminal pyroglutamate modified MCP-1 (MCP-1 N1pE)” refer to an MCP-1 peptide as hereinbefore defined wherein the N-terminal glutamine residue has been modified by glutaminyl cyclase (QC) to an N-terminal pyroglutaminyl (pGlu; pE or 5-oxo-proline) residue as described in Proost, P et al. (1996) J Leukocyte Biol. 59, 67-74).

In one embodiment, said determination comprises the following steps:
(a) determining a first concentration (cₐ) of N-terminal pyroglutamate modified MCP-1 in a biological sample;
(b) determining a second concentration (c_{d}) of total MCP-1 in said biological sample; and
(c) determining the ratio of cₐ/c_{d}, wherein the value of the first concentration (cₐ) is divided by the value of the second concentration (c_{d}).

Preferably, the ratio of cₐ/c_{d} is expressed in percent (%).

When diagnosing or monitoring the treatment of an inflammatory disease or an inflammatory associated disease, in one embodiment, the suitable range of cₐ/c_{d} ratio is 50%, 70%, 85% (i.e. a decrease by 50%, 30%, 15%).

When determining the effectiveness of a QC inhibitor in the treatment of an inflammatory disease or an inflammatory associated disease, in one embodiment, the suitable range of cₐ/c_{d} ratio is 30%, 50% and 70% (i.e. a decrease by 70%, 50%, 30%).

When assessing the profile of a QC inhibitor within a mammalian cell line, in one embodiment, the suitable range of cₐ/c_{d} ratio is 10% 30% and 50% (i.e. a decrease by 90%, 70%, 50%).

A further aspect of the invention provides ligands, such as naturally occurring or chemically synthesised compounds, capable of specific binding to the MCP-1 N1pE biomarker and MCP-1. A ligand according to the invention may comprise a peptide, an antibody or a fragment thereof, or an aptamer or oligonucleotide, capable of specific binding to the MCP-1 N1pE biomarker and MCP-1.

In one embodiment, step (a) comprises:
i) contacting a biological sample with a capture antibody specific for MCP-1,
ii) application of a detection antibody specific for N-terminal pyroglutamate modified MCP-1,
iii) detection of the resulting immune complex, and
iii) quantifying the detected N-terminal pyroglutamate modified MCP-1 complex.

In one embodiment, the capture antibody is a monoclonal antibody or a fragment thereof capable of specific binding to MCP-1. In one embodiment, the detection antibody is a monoclonal antibody or a fragment thereof capable of specific binding to the MCP-1 N1pE biomarker. In a further embodiment, the detection antibody specific for N-terminal pyroglutamate modified MCP-1 comprises an antibody as described in International Patent Application No. PCT/EP2009/60757, the MCP-1 N1pE detecting antibodies of which are incorporated herein by reference.

More preferably the detection antibody specific for N-terminal pyroglutamate modified MCP-1 is a monoclonal antibody, wherein the variable part of the light chain of said antibody has a nucleotide sequence selected from SEQ ID NOs: 33, and 41 as described in International Patent Application No. PCT/EP2009/60757, or an amino acid sequence selected from SEQ ID NOs: 34, and 42 as described in International Patent Application No. PCT/EP2009/60757.

Alternatively preferred according to the present invention is a monoclonal antibody specific for N-terminal pyroglutamate modified MCP-1, wherein the variable part of the heavy chain of said antibody has a nucleotide sequence selected from SEQ ID NOs: 35, 39 and 43 as described in International Patent Application No. PCT/EP2009/60757, or an amino acid sequence selected from SEQ ID NOs: 36, 40 and 44 as described in International Patent Application No. PCT/EP2009/60757.

Further preferred according to the present invention is a monoclonal antibody specific for N-terminal pyroglutamate modified MCP-1, wherein the variable part of the light chain of said antibody has the nucleotide sequence of SEQ ID NO: 33 as described in International Patent Application No. PCT/EP2009/60757 or the amino acid sequence of SEQ ID NO: 34 as described in International Patent Application No. PCT/EP2009/60757, and wherein the variable part of the heavy chain of said antibody has the nucleotide sequence of SEQ ID NO: 35 as described in International Patent Application No. PCT/EP2009/60757, or the amino acid sequence of SEQ ID NO: 36 as described in International Patent Application No. PCT/EP2009/60757.

Also preferred according to the present invention is a monoclonal antibody specific for N-terminal pyroglutamate modified MCP-1, wherein the variable part of the light chain of said antibody has the nucleotide sequence of SEQ ID NO: 37 as described in International Patent Application No. PCT/EP2009/60757 or the amino acid sequence of SEQ ID NO: 38 as described in International Patent Application No. PCT/EP2009/60757, and wherein the variable part of the heavy chain of said antibody has the nucleotide sequence of SEQ ID NO: 39 as described in International Patent Application No. PCT/EP2009/60757, or the amino acid sequence of SEQ ID NO: 40 as described in International Patent Application No. PCT/EP2009/60757.

Even preferred according to the present invention is a monoclonal antibody specific for N-terminal pyroglutamate modified MCP-1, wherein the variable part of the light chain of said antibody has the nucleotide sequence of SEQ ID NO: 41 as described in International Patent Application No. PCT/EP2009/60757 or the amino acid sequence of SEQ ID NO: 42 as described in International Patent Application No. PCT/EP2009/60757, and wherein the variable part of the heavy chain of said antibody has the nucleotide sequence of SEQ ID NO: 43 as described in International Patent Application No. PCT/EP2009/60757, or the amino acid sequence of SEQ ID NO: 44 as described in International Patent Application No. PCT/EP2009/60757.

In a particular preferred embodiment, the monoclonal antibody specific for N-terminal pyroglutamate modified MCP-1 is produced by a hybridoma cell line selected from the following group:

| | |
|---|---|
| 348/1D4 | (Deposit No. DSM ACC 2905); |
| 348/2C9 | (Deposit No. DSM ACC 2906); |
| 332/4B8 | (Deposit No. DSM ACC 2907); and |
| 332/4F8 | (Deposit No. DSM ACC 2908). |

In an especially preferred embodiment, the monoclonal antibody specific for N-terminal pyroglutamate modified MCP-1 is produced by a hybridoma cell line selected from 348/2C9 (Deposit No. DSM ACC 2906).

According to a further preferred embodiment, the antibody specific for N-terminal pyroglutamate modified MCP-1 can be humanised or is a chimeric antibody or is a human antibody.

Further, the antibody specific for N-terminal pyroglutamate modified MCP-1 as selected from the above-mentioned group can also be a functional variant of said group.

In the context of the present invention a “functional variant” of the antibody specific for N-terminal pyroglutamate modified MCP-1 is an antibody which retains the binding capacities, in particular binding capacities with high affinity to a MCP-1 N1pE-38 or functional variant thereof. The provision of such functional variants is known in the art and encompasses the above-mentioned possibilities, which were indicated under the definition of antibodies and fragments thereof.

In a preferred embodiment, the antibody specific for N-terminal pyroglutamate modified MCP-1 is an antibody fragment, as defined above.

In a further preferred embodiment, the antibody specific for N-terminal pyroglutamate modified MCP-1 is an antibody which has the complementarity-determining regions (CDRs) of the above-defined antibodies. Preferably, the antibody specific for N-terminal pyroglutamate modified MCP-1 can be labeled; possible labels are those as mentioned above and all those known to a person skilled in the art of diagnostic uses of antibodies in particular.

Further preferred according to the present invention is a monoclonal antibody specific for N-terminal pyroglutamate modified MCP-1 including any functionally equivalent antibody or functional parts thereof, which antibody comprises a light chain variable domain comprising an amino acid sequence that is 85%, 86%, 87%, 88%, 89%, 90%, 91%, 92%, 93%, 94%, 95%, 96%, 97%, 98% or 99% identical to a sequence selected from SEQ ID NOs: 34, 38 or 42 as described in International Patent Application No. PCT/EP2009/60757.

Even preferred according to the present invention is a monoclonal antibody specific for N-terminal pyroglutamate modified MCP-1 including any functionally equivalent antibody or functional parts thereof, which antibody comprises a heavy chain variable domain comprising an amino acid sequence that is 85%, 86%, 87%, 88%, 89%, 90%, 91%, 92%, 93%, 94%, 95%, 96%, 97%, 98% or 99% identical to a sequence selected from SEQ ID NOs: 36, 40 or 44 as described in International Patent Application No. PCT/EP2009/60757.

Moreover, the monoclonal antibody specific for N-terminal pyroglutamate modified MCP-1 including any functionally equivalent antibody or functional parts thereof, wherein the variable part of the light chain of said antibody comprises an amino acid sequence selected from SEQ ID NOs: 34, 38 and 42 as described in International Patent Application No. PCT/EP2009/60757 and/or wherein the variable part of the heavy chain of said antibody comprises an amino acid sequence selected from SEQ ID NOs: 36, 40 and 44 as described in International Patent Application No. PCT/EP2009/60757, wherein the antibody has been altered by introducing at least one, at least two, or at least 3 or more conservative substitutions into at least one of the sequences of SEQ ID NOs: 34, 36, 38, 40, 42 and 44 as described in International Patent Application No. PCT/EP2009/60757, wherein the antibody essentially maintains its full functionality.

Preferably, the antibody specific for N-terminal pyroglutamate modified MCP-1 is immobilised on a solid phase.

In one embodiment, step (b) comprises:
i) contacting a biological sample with a capture antibody specific for MCP-1,
ii) application of a detection antibody specific for MCP-1,
iii) detection of the resulting immune complex, and
iv) quantifying the captured MCP-1 complex.

In one embodiment, the capture antibody used in step i) is a monoclonal antibody or a fragment thereof capable of specific binding to MCP-1. In a further embodiment, the capture antibody specific for MCP-1 used in step i) is selected from:

polyclonal antiserum goat anti-hMCP1-AF (R&D Systems, Minneapolis, USA);
rabbit polyclonal to MCP-1 antibody ab18072 (Abcam, Cambridge, UK);
rabbit polyclonal to MCP-1 antibody ab9669 (Abcam, Cambridge, UK);
rabbit polyclonal to MCP-1 antibody ab18072 (Abcam, Cambridge, UK);
goat MCP-1 antibody (C-17): sc-1304 (Santa Cruz Biotechnology, Santa Cruz, USA);
polyclonal antiserum rabbit anti mJE (Peprotech, Hamburg, Germany);
rabbit polyclonal to mMCP-1 antibody ab9899 (Abcam, Cambridge, UK);
rabbit polyclonal to MCP-1 antibody ab7202 (Abcam, Cambridge, UK); and
rat monoclonal MCP-1 antibody (JJ5): sc-74215 (Santa Cruz Biotechnology, Santa Cruz, USA).

In a yet further embodiment, the capture antibody specific for MCP-1 used in step i) is selected from polyclonal antiserum goat anti-hMCP1-AF (R&D Systems, Minneapolis, USA).

In one embodiment, the detection antibody specific for MCP-1 used in step ii) comprises:

mouse anti hMCP-1 (Peprotech, Hamburg, Germany);
mouse monoclonal to MCP-1 antibody ab17715 (Abcam, Cambridge, UK);
mouse monoclonal MCP-1 antibody sc-32819 (Santa Cruz Biotechnology, Santa Cruz, USA);
anti mouse MCP-1 (R&D Systems, Minneapolis, Minn. USA);
hamster monoclonal MCP-1 antibody ab21397 (Abcam, Cambridge, UK);
rat monoclonal MCP-1 antibody ab8101 (Abcam, Cambridge, UK); and
rat monoclonal MCP-1 antibody (JJ5): sc-74215 (Santa Cruz Biotechnology, Santa Cruz, USA).

In one embodiment, the detection of the complex is carried out by using secondary antibodies, specifically reacting with each detection antibody.

In one embodiment, the secondary antibodies are anti-mouse antibodies or anti-rabbit antibodies, such as anti-mouse antibodies.

In one embodiment, the secondary antibodies are labeled. For diagnostic applications, the secondary antibody will typically be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Gütigen et al., Ed., Wiley-Interscience. New York, N.Y. Pubs., (1991) for example and radioactivity can be measured using scintillation counting.
(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra for example. Fluorescence can be quantified using a fluorimeter.
(c) Various enzyme-substrate labels are available. The enzyme generally catalyses a chemical alteration of the chromogenic substrate which can be measured using various techniques. For example, the enzyme may catalyze a colour change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g, firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase. β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed Langone & H. Van Vunakis), Academic Press, New York, 73: 147-166 (1981).

Examples of enzyme-substrate combinations include, for example:
(i) Horseradish peroxidase (HRPO) with hydrogen peroxidase as a substrate, wherein the hydrogen peroxidase oxidizes a dye precursor (e.g. orthophenylene diamine (OPD) or 3,3′,5,5′-tetramethyl benzidine hydrochloride (TMB));
(ii) alkaline phosphatase (AP) with para-Nitrophenyl phosphate as chromogenic substrate; and
(iii) β-D-galactosidase (β-D-Gal) with a chromogenic substrate (e.g. p-nitrophenyl-β-D-galactosidase) or the fluorogenic substrate 4-methylumbelliferyl-β-D-galactosidase.

Numerous other enzyme-substrate combinations are available to those skilled in the art.

(d) Another possible label for a detection antibody is a short nucleotide sequence. The concentration is then determined by a RT-PCR system (Imperacer™, Chimera Biotech).

Sometimes, the label is indirectly conjugated with the detection antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). Thus, indirect conjugation of the label with the antibody can be achieved.

The antibodies used in the present invention may be employed in any known assay method, such as competitive binding assays, direct and indirect sandwich assays, and immunoprecipitation assays. Zola, Monoclonal Antibodies A Manual of Techniques, pp. 147-158 (CRC Press. Inc., 1987).

Competitive binding assays rely on the ability of a labeled standard to compete with the test sample analyte for binding with a limited amount of antibody. The amount of target peptide in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

In a further embodiment, the secondary antibodies are labelled with horseradish peroxidase (HRP).

In one embodiment, the detected immune complex is quantified.

In one embodiment, the captured complexes are quantified by a quantification means selected from the group consisting of: ELISA, such as indirect ELISA, sandwich ELISA, competitive ELISA, reverse ELISA, enzyme-linked immunosorbent spot assay; flow cytometry; Multiplex Assay Systems; immunohistochemistry; immunoprecipitation; and Western Blot analysis. In a further embodiment, the captured complexes are quantified by a sandwich ELISA as quantification means. A suitable example of the sandwich ELISA method which may be used in accordance with the invention is described in Examples 5 and 11.

In one embodiment, the biological sample is selected from the group consisting of blood, serum, urine, cerebrospinal fluid (CSF), plasma, lymph, saliva, sweat, pleural fluid, synovial fluid, tear fluid, bile and pancreas secretion. In a further embodiment, the biological sample is serum. According to another preferred embodiment, said sample is a liquor, cerebrospinal fluid (CSF) or synovial fluid sample. The biological sample can be obtained from a patient in a manner well-known to a person skilled in the art. In particular, a blood sample can be obtained from a subject and the blood sample can be separated into serum and plasma by conventional methods. The subject, from which the biological sample is obtained is suspected of being afflicted with an inflammatory disease or an inflammatory associated disease and/or at risk of developing an inflammatory disease or an inflammatory associated disease.

A further aspect of the invention comprises biosensors which comprise the MCP-1 N1pE biomarker or a structural/shape mimic thereof capable of specific binding to an antibody against the MCP-1 N1pE biomarker. Also provided is an array comprising a ligand or mimic as described herein. The term “biosensor” means anything capable of detecting the presence of the MCP-1 N1pE biomarker.

Biosensors according to the invention may comprise a ligand or ligands, as described herein, capable of specific binding to the MCP-1 N1pE biomarker. Such biosensors are useful in detecting and/or quantifying the MCP-1 N1pE biomarker of the invention.

According to a third aspect of the invention, there is provided a method of determining the proportion of N-terminal pyroglutamate modified MCP-1 in relation to the total concentration of MCP-1 within a biological sample which comprises the following steps:
(a) determining a first concentration (cₐ) of N-terminal pyroglutamate modified MCP-1 in a biological sample;
(b) determining a second concentration (c_{d}) of total MCP-1 in said biological sample; and
(c) determining the ratio of cₐ/c_{d}, wherein the value of the first concentration (cₐ) is divided by the value of the second concentration (c_{d}).

It will be appreciated that the present invention provides an effective and sensitive method of measuring the proportion of N-terminal pyroglutamate modified MCP-1 in relation to the total concentration of MCP-1. In view of the fact that that glutaminyl cyclase (QC) post-translationally modifies MCP-1 to possess an N-terminal pyroglutaminyl residue, the method of the present invention therefore also finds utility as an effective screening method for assessing the ability of a test agent to affect QC activity. Thus, according to a further aspect of the invention, there is provided a method of screening for a glutaminyl cyclase (QC) inhibitor which comprises the steps of:
(a) incubating a control sample comprising MCP-1 and glutaminyl cyclase (QC) and determining the proportion of N-terminal pyroglutamate modified MCP-1 in relation to the total concentration of MCP-1;
(b) incubating a control sample with a mixture comprising MCP-1 and glutaminyl cyclase (QC) together with a glutaminyl cyclase (QC) inhibitor and determining the proportion of N-terminal pyroglutamate modified MCP-1 in relation to the total concentration of MCP-1;
   such that a reduction in the ratio of N-terminal pyroglutamate modified MCP-1:total MCP-1 in step (b) relative to step (a) is indicative of glutaminyl cyclase inhibition.

According to a further aspect of the invention, there is provided a glutaminyl cyclase (QC) inhibitor obtainable by a screening method as hereinbefore defined.

According to a further aspect of the invention, there is provided a method for measuring the effectiveness of a glutaminyl cyclase (QC) inhibitor which comprises incubating a glutaminyl cyclase (QC) inhibitor with a mixture comprising MCP-1 and glutaminyl cyclase (QC) and determining the proportion of N-terminal pyroglutamate modified MCP-1 in relation to the total concentration of MCP-1. This aspect of the invention provides the advantage of assessing the effectiveness of an already identified QC inhibitor, for example, a reduction in the rate of conversion of MCP-1 to N-terminal pyroglutamate modified MCP-1 can be assessed over a given period of time.

### Diagnostic Kits

As a matter of convenience, the antibodies used in the method of the present invention can be provided in a kit, i.e., a packaged combination of reagents in predetermined amounts with instructions for performing the diagnostic assay.

According to a further aspect of the invention, there is provided a kit for diagnosing an inflammatory disease or an inflammatory associated disease which comprises a capture antibody specific for MCP-1, a detection antibody specific for N-terminal pyroglutamate modified MCP-1, a detection antibody specific for MCP-1, and optionally, instructions to use said kit in accordance with the methods as defined hereinbefore.

Where the antibody is labeled with an enzyme, the kit will include substrates and cofactors required by the enzyme (e.g. a substrate precursor which provides the detectable chromophore or fluorophore). In addition, other additives may be included such as stabilizers, buffers (e.g. a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

The method of the invention also has industrial applicability to monitoring the efficacy of a given treatment of an inflammatory disease or an inflammatory associated disease. According to a further aspect of the invention, there is provided a method of monitoring efficacy of a therapy in a subject having, suspected of having, or of being predisposed to, an inflammatory disease or an inflammatory associated disease, comprising determining the proportion of N-terminal pyroglutamate modified MCP-1 in relation to the total concentration of MCP-1 as defined hereinbefore in a biological sample from a test subject.

According to a further aspect of the invention, there is provided a method of diagnosing or monitoring as defined hereinbefore, which comprises determining the proportion of N-terminal pyroglutamate modified MCP-1 in relation to the total concentration of MCP-1 in a biological sample taken on two or more occasions from a test subject.

According to a further aspect of the invention, there is provided a method of diagnosing or monitoring as defined hereinbefore, which comprises comparing the proportion of N-terminal pyroglutamate modified MCP-1 in relation to the total concentration of MCP-1 in the biological samples taken on two or more occasions.

In one embodiment, the inflammatory disease or inflammatory associated disease is an MCP-1-related disease, e.g. atherosclerosis, rheumatoid arthritis, asthma, delayed hypersensitivity reactions, pancreatitis, Alzheimer's disease, hyperinsulinemia and obesity, including Type II diabetes, diabetic nephropathy, colitis, lung fibrosis, renal fibrosis, gestosis, graft rejection, neuropathic pain, stroke, AIDS and tumors.

Most preferably, the inflammatory disease or inflammatory associated disease is Alzheimer's disease, or also most preferably a disease selected from atherosclerosis, rheumatoid arthritis, restenosis and pancreatitis, diabetic nephropathy, in particular Alzheimer's disease or rheumatoid arthritis.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

The present invention is further described by the following examples, which should however by no means be construed to limit the invention in any way; the invention is defined in its scope only by the claims as enclosed herewith.

### EXAMPLES OF THE INVENTION

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### MALDI-TOF Mass Spectrometry

Matrix-assisted laser desorption/ionization mass spectrometry was carried out using the Voyager De-Pro (Applied Biosystems, Darmstadt) with a linear time of flight analyzer. The instrument was equipped with a 337 nm nitrogen laser, a potential acceleration source and a 1.4 m flight tube. Detector operation was in the positive-ion mode. Samples (5 μl) were mixed with equal volumes of the matrix solution. For matrix solution sinapinic acid was used, prepared by solving 20 mg sinapinic acid (Sigma-Aldrich) in 1 ml acetonitrile/0.1% TFA in water (1/1, v/v). A small volume (≈1 μl) of the matrix-analyte-mixture was transferred to a probe tip.

For long-term testing of Glu¹-cyclization, MCP-1 peptides were incubated in 100 μl 0.1 M sodium acetate buffer, pH 5.2 or 0.1 M Bis-Tris buffer, pH 6.5 at 30° C. Peptides were applied in 0.15 mM to 0.5 mM concentrations, and 0.2 U QC was added. At different times, samples were removed from the assay tube, peptides extracted using ZipTips (Millipore) according to the manufacturer's recommendations, mixed with matrix solution (1:1 v/v) and subsequently the mass spectra recorded. Negative controls contained either no QC or heat deactivated enzyme. For the inhibitor studies the sample composition was the same as described above, with the exception of the inhibitory compound added.

### Example 2

### Proteolytic Degradation of Human MCP-1(1-76) by Dipeptidyl-Peptidase 4 (DP4), Aminopeptidase P, and by Proteases Present in Human Serum

### N-Terminal Degradation of MCP-1 by Recombinant Human DP4 in Absence and Presence of a QC-Specific Inhibitor

Recombinant human MCP-1(1-76) (SEQ ID NO: 1) starting with an N-terminal glutamine (Peprotech) was dissolved in 25 mM Tris/HCl pH 7.6 in a concentration of 10 μg/ml. The MCP-1 solution was either pre-incubated with recombinant human QC (0.0006 mg/ml) for 3 h at 30° C. and subsequently incubated with recombinant human DP4 (0.0012 mg/ml) at 30° C. or incubated with DP4 without prior QC application. In addition, the incubation of Gln¹-MCP-1 with recombinant human QC was carried out in the presence of 10 μM of 1-(3-(1H-imidazol-1-yl)propyl)-3-(3,4-dimethoxyphenyl)thiourea hydrochloride. Resulting DP4 cleavage products were analyzed using Maldi-TOF mass spectrometry after 0 min, 15 min, 30 min, 1 h, 2 h and 4 h.

### N-Terminal Degradation by Recombinant Human Aminopeptidase P

Human recombinant MCP-1 carrying an N-terminal glutaminyl instead of a pyroglutamyl residue (Peprotech) was dissolved in 25 mM Tris/HCl, pH 7.6 in a concentration of 10 μg/ml.

MCP-1 was incubated with 30 μg/ml Aminopeptidase P (R&D Systems) at 30° C. Gln¹-MCP-1 was either used without pGlu-modification or was pre-incubated with recombinant human QC (6 μg/ml) for 3 h at 30° C. in order to generate pGlu.

Resulting Aminopeptidase P cleavage products were analyzed using Maldi-TOF mass spectrometry after 0 min, 15 min, 30 min, 1 h, 2 h, 4 h and 24 h.

### N-Terminal Degradation of Human MCP-1 in Human Serum

Human recombinant MCP-1 carrying an N-terminal glutaminyl residue (Peprotech) was dissolved in 25 mM Tris/HCl, pH 7.6, in a concentration of 100 μg/ml. MCP-1 was either pre-incubated with recombinant human QC (0.006 mg/ml) for 3 h at 30° C. and subsequently incubated with human serum at 30° C. or incubated with human serum without addition of QC. The cleavage products were analyzed using Maldi-TOF mass spectrometry after 0 min, 10 min, 30 min, 1 h, 2 h, 3 h 5 h and 7 h for Gln¹-MCP-1 and 0 min, 30 min, 1 h, 2 h, 3 h 5 h, 7 h and 24 h for pGlu¹-MCP-1.

### Example 3

### Degradation of Human MCP-2, MCP-3 and MCP-4 N-Terminal Degradation of Human MCP-2 by DP4

Human recombinant MCP-2 (SEQ ID NO: 11) carrying an N-terminal glutaminyl instead of a pyroglutamyl residue (Peprotech) was dissolved in 25 mM Tris/HCl, pH 7.6, in a concentration of 10 μg/ml. MCP-2 was either pre-incubated with recombinant human QC (0.0006 mg/ml) for 3 h at 30° C. and subsequently incubated with recombinant human DP4 (0.0012 mg/ml) at 30° C. or incubated with recombinant human DP4 (0.0012 mg/ml) without pre-incubation with QC. Resulting DP4 cleavage products were analyzed using Maldi-TOF mass spectrometry after 0 min, 15 min, 30 min, 1 h, 2 h, 4 h and 24 h.

### N-Terminal Degradation of Human MCP-3 by DP4

Human recombinant MCP-3 (SEQ ID NO: 12) carrying an N-terminal glutaminyl instead of a pyroglutamyl residue (Peprotech) was dissolved in 25 mM Tris/HCl, pH 7.6, in a concentration of 10 μg/ml. MCP-3 was either pre-incubated with recombinant human QC (0.0006 mg/ml) for 3 h at 30° C. and subsequently incubated with recombinant human DP4 (0.00012 mg/ml) at 30° C. or incubated with recombinant human DP4 (0.00012 mg/ml) without prior QC application. Resulting DP4 cleavage products were analyzed using Maldi-TOF mass spectrometry after 0 min, 15 min, 30 min, 1 h, 2 h, 4 h and 24 h.

### N-Terminal Degradation of Human MCP-4 by DP4

Human recombinant MCP-4 (SEQ ID NO: 13) carrying an N-terminal glutaminyl instead of a pyroglutamyl residue (Peprotech) was dissolved in 25 mM Tris/HCl, pH 7.6, in a concentration of 10 μg/ml. MCP-4 was either pre-incubated with recombinant human QC (0.0006 mg/ml) for 3 h at 30° C. and subsequently incubated with recombinant human DP4 (0.00006 mg/ml) at 30° C. or incubated with recombinant human DP4 (0.00006 mg/ml) without prior QC application. Resulting DP4 cleavage products were analyzed using Maldi-TOF mass spectrometry after 0 min, 15 min, 30 min, 1 h, 2 h, 4 h and 24 h.

### Example 4

### Chemotactic Potency of Different N-Terminal Variants of Human MCP-1, MCP-2, MCP-3, MCP-4

### TransWell Chemotaxis Assay

The chemotaxis assay was performed using 24 well TransWell plates with a pore size of 5 μm (Corning). THP-1 cells were suspended in RPMI1640 in a concentration of 1*10⁶ cells/100 μl and applied in 100 μl aliquots to the upper chamber. Cells were allowed to migrate towards the chemoattractant for 2 h at 37° C. Subsequently, cells from the upper chamber were discarded and the lower chamber was mixed with 50 μl 70 mM EDTA in PBS and incubated for 15 min at 37° C. to release cells attached to the membrane. Afterwards, cells migrated to the lower chamber were counted using a cell counter system (Schärfe System). The chemotactic index was calculated by dividing cells migrated to the stimulus from cells migrated to the negative control.

### Chemotactic Potency of N-Terminal Variants of Human MCP-1

MCP-1 starting with glutamine 1 (Gln¹-MCP-1) (Peprotech) was incubated with recombinant human QC to generate pGlu¹-MCP-1, or with human recombinant DP4 to generate Asp³-MCP-1. Concentrations of 1, 5, 10, 50, 100, 500 and 1000 ng/ml of the generated MCP-1 variants were tested using the THP-1 chemotaxis assay (n=3).

### Chemotactic Potency of Human MCP-1 in Absence or Presence of a QC-Inhibitor

MCP-1 with N-terminal glutamine (Gln¹-MCP-1) (Peprotech) was incubated with recombinant human QC and DP4 (Gln¹-MCP-1+QC+DP4), human recombinant DP4 alone (Gln¹-MCP+DP4) and with recombinant human QC in combination with 10 μM of QC-inhibitor 1-(3-(1H-imidazol-1-yl)propyl)-3-(3,4-dimethoxyphenyl)thiourea hydrochloride and DP4 (Gln¹-MCP-1+QC+QCI+DP4). Concentrations of 1, 5, 10, 50, 100, 500 and 1000 ng/ml of generated MCP-1 variants were tested using chemotaxis assay (n=3).

### Comparison of the Chemotactic Potency of Variants of Human MCP-1, MCP-2, MCP-3 and MCP-4 Possessing an N-Terminal Glutaminyl or Pyroglutamyl Residue.

Human MCP-1, MCP-2, MCP-3 and MCP-4 with an N-terminal glutamine (Peprotech) or pyroglutamyl-residue (incubation of Gln¹-MCPs with human recombinant QC at a dilution of 1:100 for 2 h at 30° C.) were tested for chemotactic potency. Concentrations of 1, 5, 10, 50, 100, 500 and 1000 ng/ml of a particular MCP were tested using chemotaxis assay (n=3).

Comparison of the Chemotactic Potency of Variants of Human MCP-1, MCP-2, MCP-3 and MCP-4 Possessing an N-Terminal Glutaminyl Residue with the Respective DP4 Cleavage Product

The human MCP-1, MCP-2, MCP-3 and MCP-4 starting with an N-terminal glutamine (Peprotech) was directly applied to the chemotaxis assay and compared to chemotactic potency of the DP4 cleavage products of MCP-1, MCP-2, MCP-3 and MCP-4. For the generation of the DP4 cleavage product, the respective MCPs were incubated with human recombinant DP4 at a 1:100 dilution for 2 h at 30° C. prior to assay. Concentrations of 1, 5, 10, 50, 100, 500 and 1000 ng/ml of a particular MCP were tested using chemotaxis assay (n=3).

### Example 5

### Establishment of an Indirect Sandwich ELISA for the Quantitative Detection of Total Human MCP-1 (hMCP-1) and Human MCP-1 with an N-Terminal Pyroglutamate (hMCP-1 N1pE)

To capture human MCP-1, commercially available polyclonal antiserum goat anti-hMCP1-AF (R&D Systems, Minneapolis, USA) as capture antibody which specifically binds human MCP-1 was diluted in PBS to 250 ng/ml and immobilized in polystyrene 96-well microtitre plates overnight at 4° C. Thereafter, blocking occurred for 2 hours at room temperature with ELISA Blocker (Thermo Fisher Scientific, Waltham, USA). For preparation of the standard curve recombinant hMCP-1 was incubated with recombinant human Glutaminyl Cyclase (QC) in order to obtain hMCP-1 N1pE. The recombinant hMCP-1 N1pE standard peptide was serially diluted with ELISA Blocker from 1000 pg/ml down to 15.63 pg/ml and added to the wells in duplicate. Two wells filled with ELISA Blocker represent the standard curve value 0 pg/ml. After an incubation period of 2 hours at room temperature, plates were washed at least three times with TBS-T. For detection of hMCP-1 N1pE, the MCP-1 N1pE antibody clone 348-2C9 together with HRP-conjugated anti mouse antibody were both diluted in blocking buffer to final concentrations of 500 ng/ml. For detection of hMCP-1, the antibody mouse anti hMCP-1 (Peprotech, Hamburg, Germany) together with HRP-conjugated anti mouse antibody were also both diluted in blocking buffer to final concentrations of 500 ng/ml. The detection antibody/conjugate solutions were incubated for 2 hours at room temperature. Following several washes with TBS-T a colour reaction with commercially available HRP substrate TMB (SureBlue Reserve TMB Microwell Peroxidase Substrate (1-component), KPL, Gaithersburg, USA) was performed (30 minutes incubation at room temperature in the dark) and subsequently stopped by the addition of 1,2N H₂SO₄. Absorption at 450/540 nm was determined by a Tecan Sunrise plate reader.

### Example 6

### Evaluation of the Influence of the Standard Peptide Cyclization State to the Total hMCP-1 ELISA

In order to exclude an influence of the cyclization state of the standard peptide to the total hMCP-1 ELISA, the detection of cyclized and not cyclized recombinant human MCP-1 was compared.

The ELISA protocol corresponds to Example 5, for preparation of the standard curves hMCP-1 was incubated with or without QC.

### Example 7

### Determination of the hMCP-1 N1pE/hMCP-1 Ratio in Cell Culture Supernatants of Stimulated NHDF Cells by ELISA

Following an inflammatory stimulus, the expression of hMCP1 is enhanced in Human Normal Dermal Fibroblasts (NHDF). Hence, the amount of hMCP-1, as well as MCP-1 N1pE, should increase after application of Oncostatin M (OSM) and Interleukin 1β (IL1β) to NHDF. To prove this, OSM and IL1β stimulated NHDF cell culture supernatants were subjected to two ELISA analyses. The amount of hMCP-1, and the portion of hMCP-1 N1pE were analyzed.

Quantitative detection of hMCP-1 and hMCP-1 N1pE occurred according to the protocol in Example 5. The NHDF cell culture supernatants were diluted in blocking buffer before addition to the wells. NHDF have been stimulated with 10 ng/ml OSM and IL1β over 14 days, reapplication of the cytokines occurred after 7 days. The cell culture supernatants were analyzed at different time points in order to examine time dependency of hMCP-1 and hMCP-1 N1pE secretion.

### Example 8

### Determination of the hMCP-1 N1pE/hMCP-1 Ratio in Normal Human Dermal Fibroblasts Treated with Glutaminyl Cyclase Inhibitor (QCI)

Example 7 shows, that hMCP-1 as well as hMCP-1 N1pE expression is enhanced in NHDF after an inflammatory stimulus. Since Glutaminyl Cyclase (QC) catalyses the formation of N-terminal pyroglutamate residues, the inhibition of QC should result in decreased hMCP-1 N1pE levels. To prove this, NHDF were stimulated with OSM and IL1β and treated with or without QCI simultaneously.

NHDF have been stimulated with 10 ng/ml OSM, IL1β and simultaneously treated with or without 10 μM QCI for 6 days. Cytokine and inhibitor application occurred once at day 0. In order to examine the influence of QCI on hMCP-1 and hMCP-1 N1pE level, the cell culture supernatants were analyzed at different time points according to the ELISA protocol in Example 7.

### Example 9

### Determination of the hMCP-1 N1pE/hMCP-1 Ratio in a Human Lung Carcinoma Cell Line (A549) Treated with Different Concentrations of QCI

Example 8 shows that application of QCI reduces hMCP-1 N1pE level in NHDF. In order to analyse the QCI concentration dependent reduction of hMCP-1 N1pE, the carcinoma human alveolar basal epithelial cell line A549 was treated with different concentrations of QCI.

A549 cells were stimulated for 24 h with 10 ng/ml TNFα and IL1β. Furthermore, QCI was applied in different concentrations to the cells. After 24 h cell culture supernatants were analyzed according to the protocol in Example 7.

### Example 10

### Spike and Recovery of hMCP1 and hMCP1 N1pE in Human Serum

In order to validate the quantitative detection of hMCP-1 and hMCP-1 N1pE in human serum, Spike and Recovery experiments were performed.

The ELISA protocol corresponds to Example 5, except the usage of FBS, 0.05% Tween, 10% FBS for blocking and dilution steps. For validation of Spike and Recovery various levels of recombinant hMCP-1 and hMCP-1 N1pE were spiked in human serum. Recovery was calculated by subtracting the value measured in the unspiked serum sample from the spiked samples.

### Example 11

### Establishment of an Indirect Sandwich ELISA for the Quantitative Detection of Total Mouse MCP-1 (mMCP-1) and Mouse MCP-1 with an N-Terminal Pyroglutamate (mMCP-1 N1pE)

Examples 5-10 describe the quantitative detection of recombinant and native human MCP-1/MCP-1 N1pE. In order to analyse mMCP-1 and mMCP-1 N1pE level in mouse samples, assays needed to developed for the quantification of mouse MCP-1/MCP-1 N1pE. Since the MCP-1 N1pE antibody clone 348-2C9 cross reacts with mouse MCP-1 N1pE, this antibody was used for the establishment of an indirect Sandwich ELISA for the detection of mMCP1 N1pE. In order to distinguish between both forms of the cytokine, a comparable indirect Sandwich ELISA was developed for the detection of total mMCP-1.

To capture mouse MCP-1, commercially available polyclonal antiserum rabbit anti mJE (Peprotech, Hamburg, Germany) as capture antibody which specifically binds mouse MCP-1 was diluted in PBS to 500 ng/ml and immobilized in polystyrene 96-well microtitre plates over 4-7 nights at 4° C. Thereafter, blocking occurred for 2 hours at room temperature with ELISA Blocker (Thermo Fisher Scientific, Waltham, USA). For preparation of the standard curve recombinant mMCP-1 was incubated with mouse Glutaminyl Cyclase (QC) in order to obtain mMCP-1 N1pE. The recombinant mMCP-1 N1pE standard peptide was serially diluted with ELISA Blocker from 1950 pg/ml down to 19.5 pg/ml and added to the wells in duplicate. Two wells filled with ELISA Blocker represent the standard curve value 0 pg/ml. After an incubation period of 2 hours at room temperature, plates were washed at least three times with TBS-T. For detection of mMCP-1 N1pE, the MCP-1 N1pE antibody clone 348-2C9 together with HRP-conjugated anti mouse antibody were both diluted in ELISA Blocker to final concentrations of 500 ng/ml. For detection of mMCP-1, the antibody rat anti mouse MCP-1 (R&D Systems, Minneapolis, Minn. USA) Goat polyclonal to MCP-1 (MCP-1 (M-18):sc-1784 (Santa Cruz) together with HRP-conjugated anti rat antibody anti Goat IgG Peroxidase Conjugate (R&D Systems, Minneapolis, Minn. USA) were also both diluted in blocking buffer to final concentrations of 250 ng/ml (200 ng/ml respectively 1 μg/ml. The detection antibody/conjugate solutions were incubated for 2 hours at room temperature. Following several washes with TBS-T a colour reaction with commercially available HRP substrate TMB (SureBlue Reserve TMB Microwell Peroxidase Substrate (1-component), KPL, Gaithersburg, USA) was performed (30 minutes incubation at room temperature in the dark) and subsequently stopped by the addition of 1,2N H₂SO₄. Absorption at 450/540 nm was determined by a Tecan Sunrise plate reader.

### Example 12

### Evaluation of the Influence of the Standard Peptide Cyclization State to the Total mMCP-1 ELISA

Example 6 demonstrates no influence of the human standard peptide hMCP-1 cyclization state on the total hMCP-1 ELISA. In order to prove this for the murine total MCP-1 ELISA a comparison of the quantification of cyclized and not cyclized recombinant mouse MCP-1 in the total mMCP-1 ELISA was performed. The ELISA protocol corresponds to Example 6, for preparation of the standard curves mMCP1 was incubated with or without QC.

### Example 13

### Determination of the mMCP-1 N1pE/mMCP-1 Ratio in a LPS Stimulated Murine Macrophage Cell Line RAW 264.7 Treated with Different Concentrations of QCI

Example 9 shows that application of QCI reduces the ratio of human MCP1 N1pE/human MCP-1 in a concentration dependent manner in stimulated A549. In order to analyse the effect of QCI on the ratio of mouse MCP-1 N1pE/mouse MCP-1, the mouse macrophage cell line RAW 264.7 was stimulated with LPS in the absence or presence of increasing concentrations of the QC inhibitor QCI. RAW 264.7 were stimulated for 24 h with 10 ng/ml LPS and treated with different QCI concentrations. After 24 h cell culture supernatants were analyzed according to the protocol in Example 9. Cell culture supernatants were diluted 1:1000 in blocking buffer before addition to the wells.

### Example 14

### Cross Validation of mMCP-1 N1pE Level in RAW 264.7 Cell Culture Supernatant by Western Blot Analysis

Stimulation of RAW 264.7 and inhibitor application occurred according to Example 9. For Western Blot analysis, proteins of cell culture supernatants were separated by application to a SDS gel electrophoresis. Separated proteins were electrically transferred to nitrocellulose membranes. Membranes were blocked for one hour with TBST-M (=TBST+5% skimmed milk) at room temperature with gentle shaking. Antibody incubation occurred over night at 4° C. on a rocking platform with the detection antibody for total mMCP-1 (Rat anti mouse MCP-1, R&D Systems) diluted to 1 μg/ml in equal volumes of TBST-M or the detection antibody for mMCP-1 N1pE (clone 332-4B8), respectively. Secondary anti-mouse and anti-rat antibody conjugated with horseradish peroxidase were used for signal detection, following standard procedures.

### Example 15

### Determination of the mMCP-1 N1pE/mMCP-1 Ratio in Mice Treated with Thioglycollate and Different Concentrations of QCI

Example 13 showed that application of QCI decreases the ratio of mMCP-1 N1pE/mMCP-1 in a mouse cell culture model. To prove this result in vivo, the ratio of mMCP-1 N1pE/mMCP-1 was measured in an acute inflammatory mouse model after application of QCI. Beyond the determination of the mMCP-1 N1pE/mMCP-1 ratio, the effect of decreased mMCP-1 N1pE concentrations on monocyte infiltration was investigated.

Thioglycollate was injected intraperitoneal in mice. Different concentrations of QCI were applied intraperitoneal 30 minutes before thioglycollate application. After 4 h peritoneal lavage fluid was performed by flushing the peritoneum with 8 ml PBS buffer. Peritoneal lavage fluids were subjected to ELISA analyses according to the protocol in Example 11 to determine mMCP1 and mMCP1 N1pE level in the peritoneum. Samples were diluted 1:5 in blocking buffer before addition to the wells. Infiltrated monocytes were counted by FACS analysis via double staining of 7/4 and Ly6G antigens.

### Example 16

### Determination of the mMCP-1 N1pE/mMCP-1 Ratio in Fluid Mouse Samples

Example 11 describes the quantitative detection of recombinant and native mMCP-1/MCP-1 N1pE. In order to analyse the mMCP-1 N1pE level in fluid mouse samples without potential cross reactivity of anti mouse IgG-HRP-conjugate, biotinylation of the MCP-1 N1pE antibody clone 348-2C9 was accomplished.

### Example 17

### Isothermal Titration Calorimetry Measurement of the Binding Affinity of Anti-MCP-1 N1pE Antibodies

The binding affinities of anti-MCP-1 N1pE antibodies (MCP-1 N1pE antibody 2C9 and biotinylated MCP-1 N1pE antibody 348/2C9) to the antigen hMCP-1(1-38) were determined usingVP-ITC microcalorimeter (MicroCal). Both antibody clones and the MCP-1 (1-38) peptide were dialyzed against 2 liter 150 mM NaCl, 25 mM Na₂HPO₄, 25 mM KH₂PO₄, 2 mM EDTA pH 7.4 overnight at 4° C. to ensure the same buffer conditions and avoid background heat by protonation events. Afterwards, the concentrations of the antibodies and the peptide and the respective extinction coefficient were calculated from absorbance at 280 nm. For the titration experiments, MCP-1 N1pE antibody 2C9 and MCP-1(1-38) were used at concentrations of 1.87 μM and 29.19 μM, respectively. The binding heat was recorded at 20° C. by titration of 29 injections of 10 μl of MCP-1(1-38) into the anti-MCP-1 N1pE antibody solution. The heat development of the dilution of the MCP-1(1-38) peptide was determined by titration into the dialysis buffer using the conditions and instrument setup. Afterwards, the data were analyzed by means of the MicroCal ORIGIN software. The calculated binding heat was corrected by the heat of the peptide dilution. The resulting curve was fitted by the “One Set of Sites” binding model and the stoichiometry, dissociation constant, reaction enthalpy, reaction entropy, were calculated.

### Example 18

### Measurement of Human MCP-1 and Human MCP-1 N1pE in CSF and Serum Samples

For the measurement of hMCP-1 and hMCP-1 N1pE in CSF and serum samples, the following protocol was used:
dilution of Goat-anti hMCP-1 antibody (R&D systems) in PBS to 250 ng/ml
addition of 100 μl per well of diluted antibody on Maxisorp 96-well plates (Nunc)
sealing of plate and incubation overnight at 4° C.
removing of antibody solution
blocking of surface by addition of 200 μl per well PBS/10% (v/v) FBS/0.05% (v/v) Tween-20, sealing of plate and incubation at room temperature for 2 h
cyclization of standard peptide:
   16.5 μl PBS
   2 μl hCCL2 (100 mg/ml)
   1 μl 22% BSA
   0.5 μl hQC
   >incubation for 1 h at 37° C.
dilution of cyclized standard peptide (10 μg/ml) in PBS/10% (v/v) FBS/0.05% (v/v) Tween-20 down to 10 ng/ml and finally to 1000 pg/ml, 500 pg/ml, 250 pg/ml . . . 15.6 pg/ml
1:4 dilution of serum or CSF samples in PBS/10% (v/v) FBS/0.05% (v/v) Tween-20 and transfer into deep well plates
washing of plate 3-times with TBS/0.05% (v/v) Tween-20, removing of wash buffer
transfer of samples and standard peptide solution from deep well plate to ELISA plate, 100 μl per well
sealing of plate and incubation at room temperature for 2 h
pre incubation of antibody 348/2C9 with anti-mouse IgG-HRP (KPL) for 15 min at RT, afterwards dilution of premix in PBS/10% (v/v) FBS/0.05% (v/v) Tween-20 down to 500 ng/ml 348/2C9 and 1 μg/ml anti-mouse-IgG-HRP
pre incubation of total hMCP-1 antibody (Biolegends) with anti-mouse IgG-HRP (KPL) for 15 min at RT, afterwards dilution of premix in PBS/10% (v/v) FBS/0.05% (v/v) Tween-20 down to 500 ng/ml 2C9 and 1 μg/ml anti-mouse-IgG-HRP
washing of plate 3-times with TBS/0.05% (v/v) Tween-20, removing of wash buffer
addition of antibody solutions to plate, 100 μl per well
sealing of plate and incubation at room temperature for 2 h
washing of plate 3-times with TBS/0.05% (v/v) Tween-20, removing of wash buffer
addition of chromogen solution (SureBlue) to plate, 100 μl per well
incubation of plate in the dark at RT for 30 min
stopping the reaction by addition of 50 μl 1.2 N H2SO4 per well
measuring absorbance at 450 nm/540 nm at TECAN Sunrise

### RESULTS AND DISCUSSION

To date, the measurement of MCP-1 levels was achieved by a number of different methods mainly based on ELISA assays.

Therefore a number of different MCP-1 antibodies have been developed for detection of total MCP1 from biological sources. They have been proven to be functional in Western Blot, for capture and detector application in ELISA's, for Intracellular Flow Cytometry (ICFC), Enzyme Linked Immunospot assay (ELISPOT), Bio-Plex cytokine assay (xMAP technology), for immune histochemical (IHC), for immunoprecipitation (IP) neutralization of receptor binding and other approaches. Consequently antibodies and ELISA-kits from different manufactures are available e.g.: Abcam, RnD systems, Bio-Rad Laboratories, Bio Source Int., IBL America, santa cruz biotechnology inc., LINCO Research Inc., Upstate, RayBiotech Inc., Enzo Biochem Inc., PeproTech, Lifespan Biosciences and others.

All of these antibodies and methods share a global disadvantage:

They fail to detect the integrity and therefore the receptor activation functionality of the MCP-1 chemokine.

Truncation of the first N-terminal amino acid residue or of the N-terminal Gln-Pro dipeptide decreases the receptor activity (CCR2) of the MCP1 chemokine by at least two orders of magnitude.

The occurrence of high concentrations of the proline specific exopeptidase dipeptidyl peptidase 4 (DP4, DPP4, CD26) rapidly decrease the level of N-terminal unmodified CCL2 within the circulation. This cytokine deactivation by DP4-mediated N-terminal truncation is totally abolished if the glutaminyl residue is posttranslational converted to pyroglutamate.

Furthermore, N-terminal degradation, usually not monitored by means of existing MCP-1 assays causes MCP-1 species with opposite characteristics:

Residues 7-10 were essential for receptor desensitization, but were not sufficient for function, and the integrity of residues 1-6 were required for functional activity. A peptide corresponding to MCP-1, 1-10 lacked detectable receptor-binding activities, indicating that residues 1-10 are essential for MCP-1 function, but that other residues are also involved. Several truncated analogues, including 8-76, 9-76, and 10-76, desensitized MCP-1-induced Ca²⁺ induction, but were not significantly active. These analogues were antagonists of MCP-1 activity with the most potent being the 9-76 analogue (ICs0=20 nM). The 9-76 specifically bound to MCP-1 receptors with a Ka of 8.3 μM, which was threefold higher than MCP-1 (Kd 2.8 nM). The 9-76 analogue desensitized the Ca²⁺ response to MCP-1 and MCP-3, but not to other CC chemokines, suggesting that it is MCP receptor specific (Gong, J.-H. and Clark-Lewis, I. (1995) J Exp. Med. 161 631-40).

### MCP Sequence Alignments

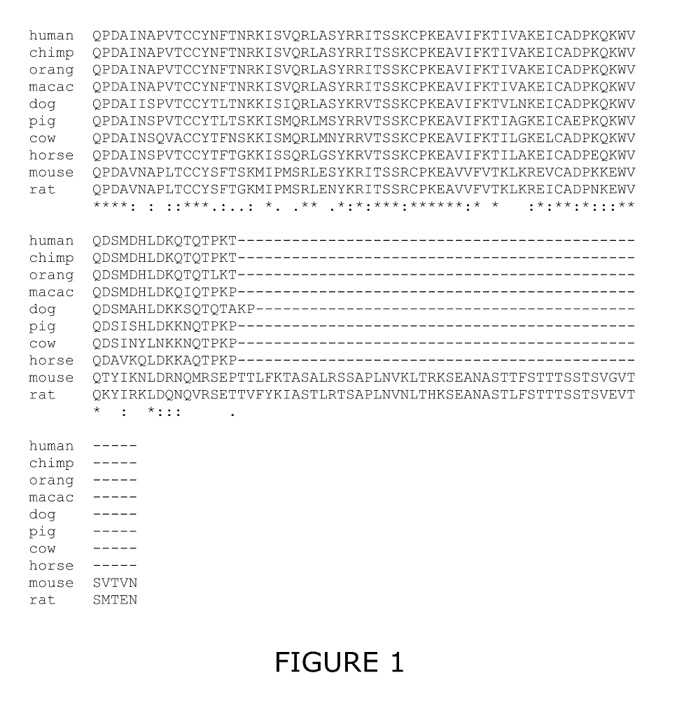

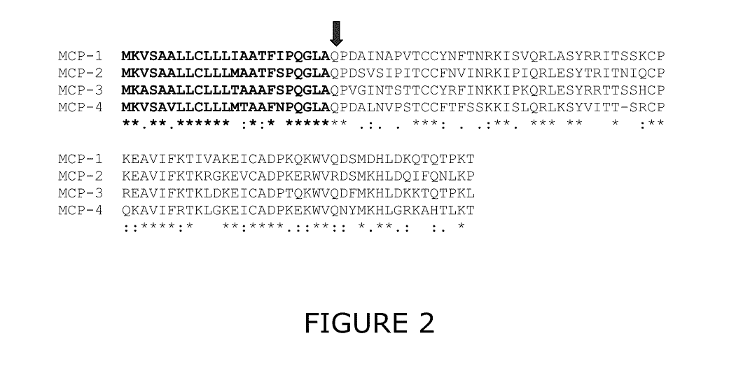

A sequence alignment of mature MCP-1 from 8 mammalian species (FIG. 1) demonstrates an overall identity of 46% and a similarity of 79%, within the first 76 amino acid residues. Especially the first four N-terminal amino acid residues are absolutely conserved ensuring the receptor agonistic/antagonistic action. A comparison of the different human MCP proteins (FIG. 2) reveals the occurrence of a N-terminal glutamine in the case of every mature protein. Due to the different receptor specificity, the adjacent amino acid residues are not conserved. But the basic principle of a QC accessible N-terminal glutamine residue together with a DP4 cleavable glutamine-proline motif remains conserved.

### Investigations on the Proteolytic Degradation of Human MCP-1(1-76)

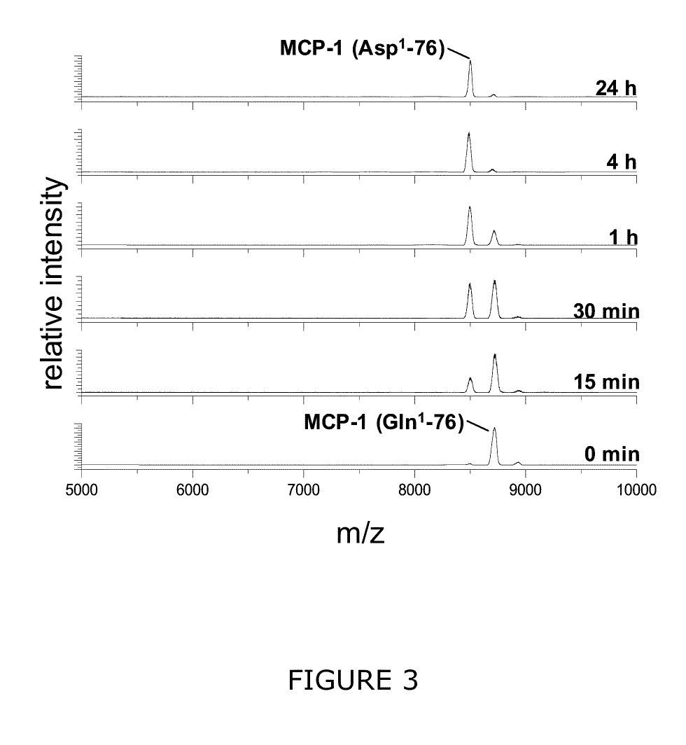

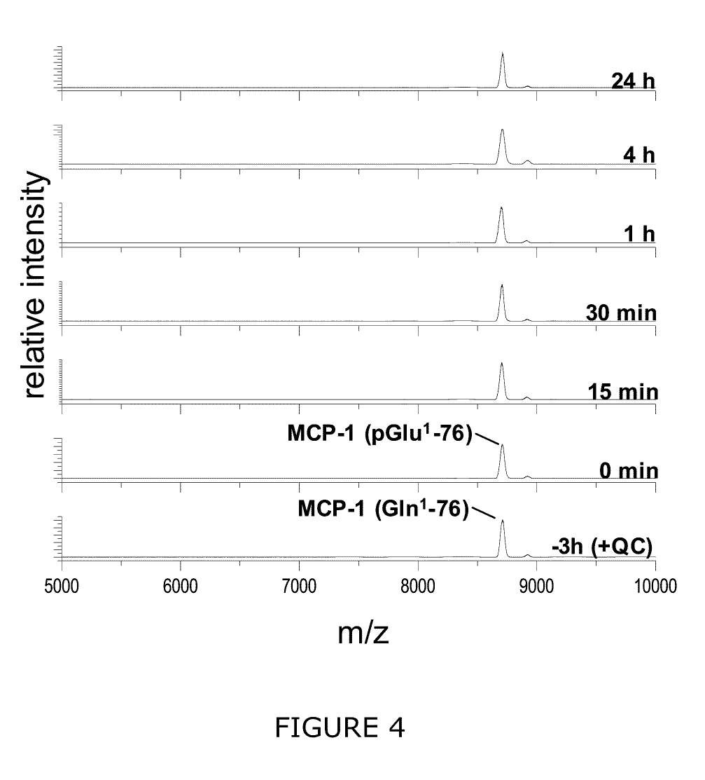

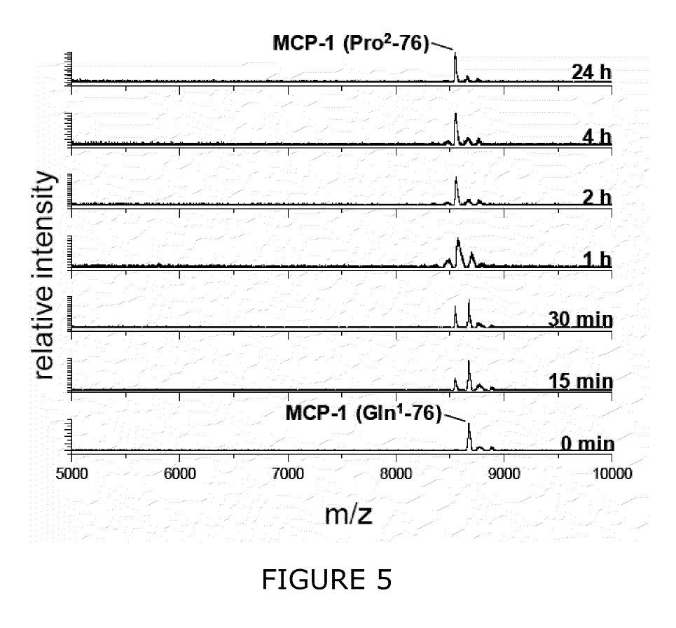

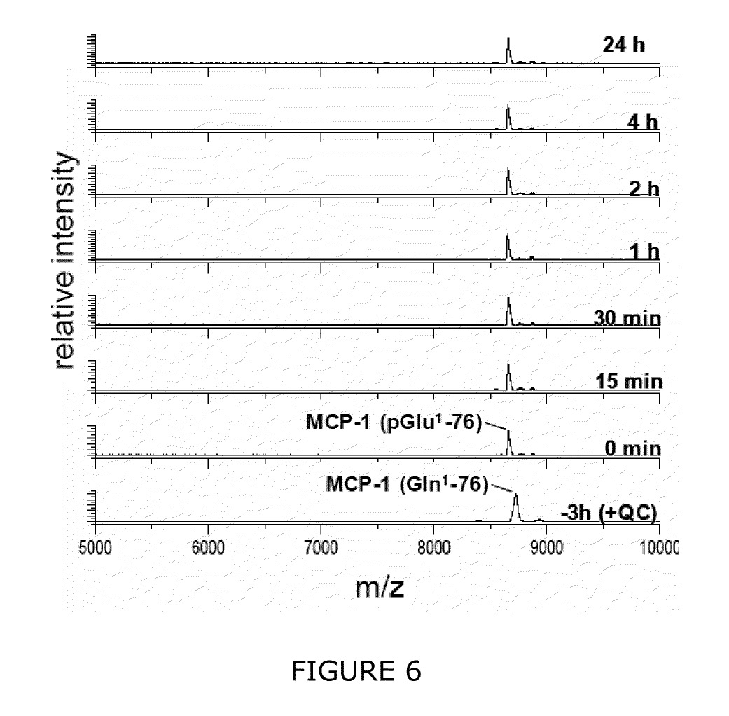

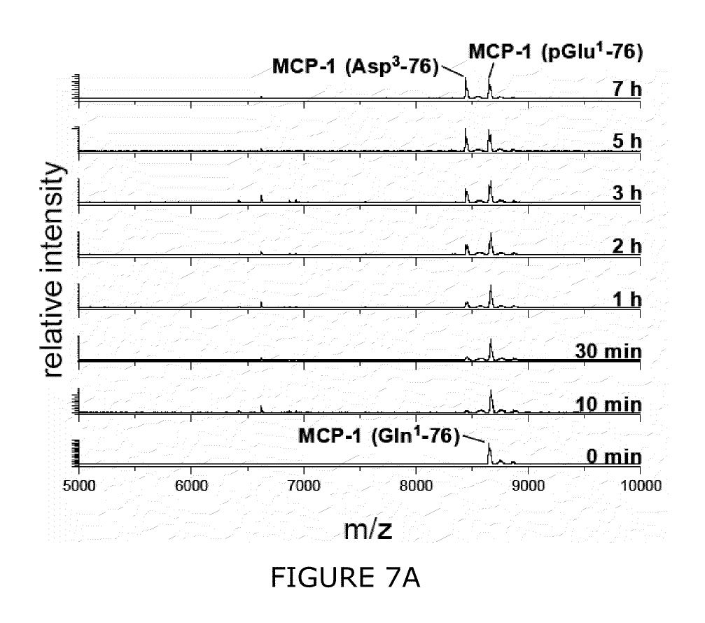

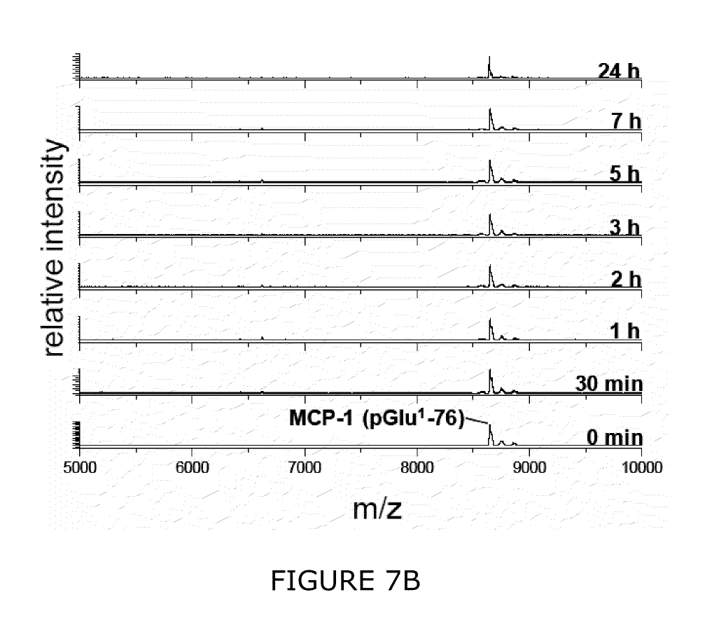

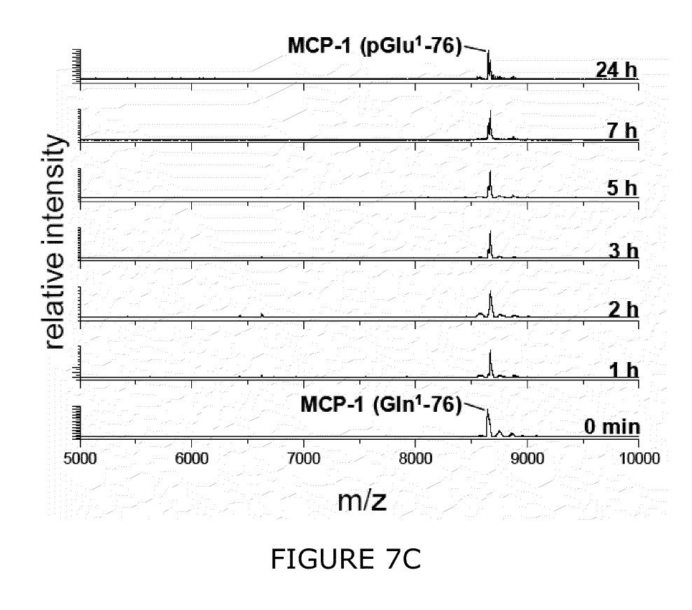

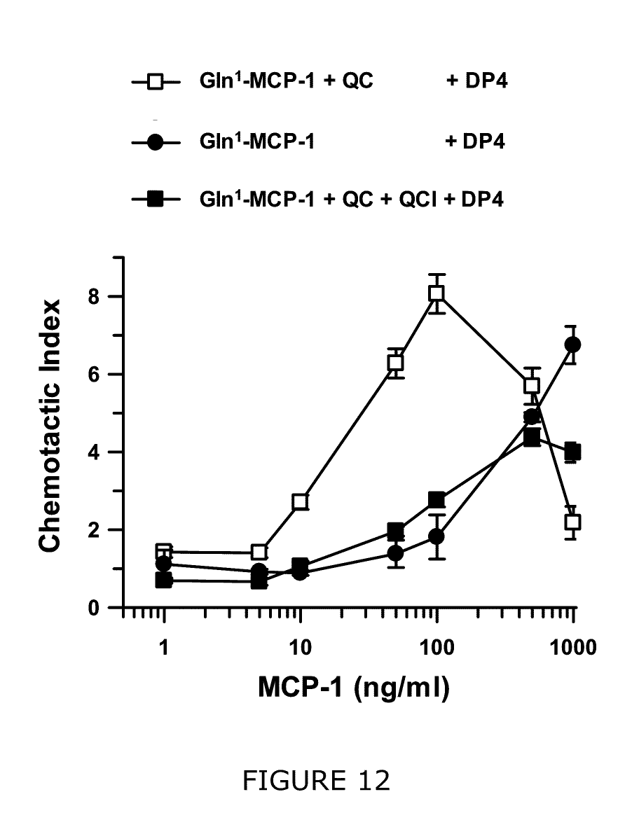

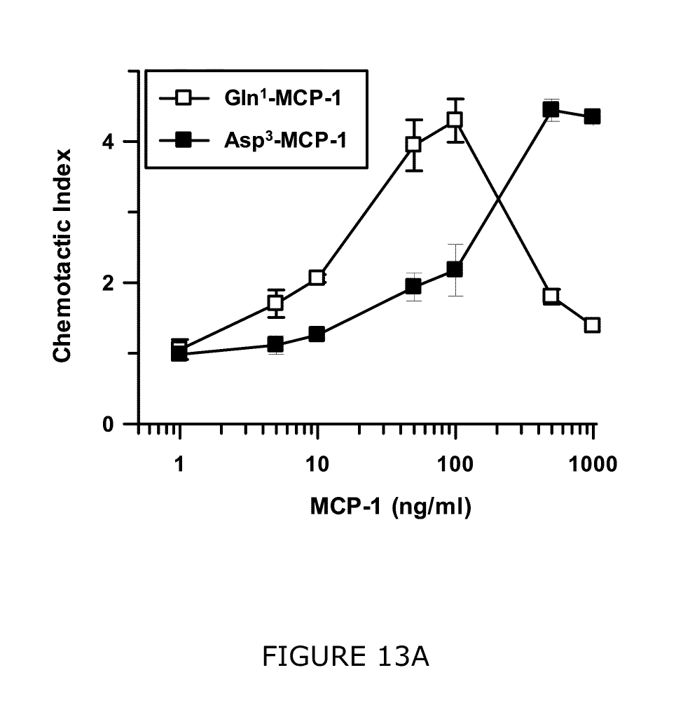

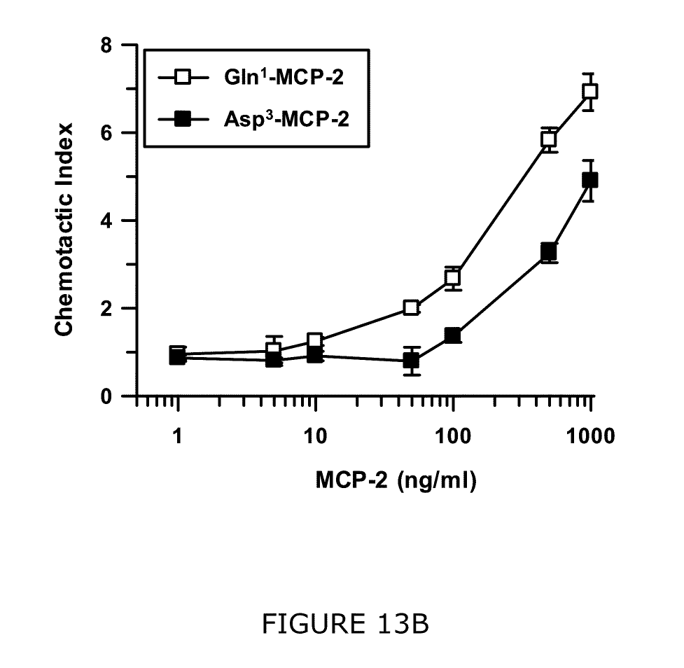

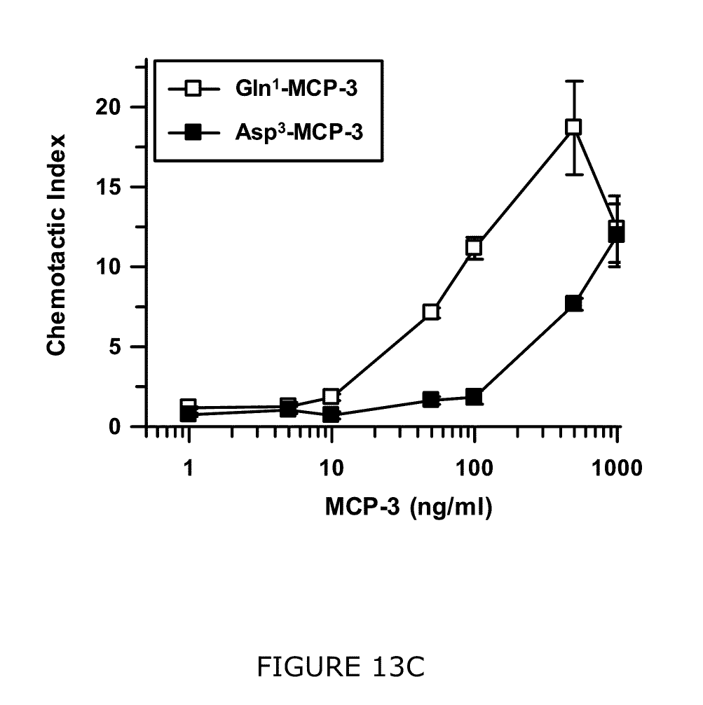

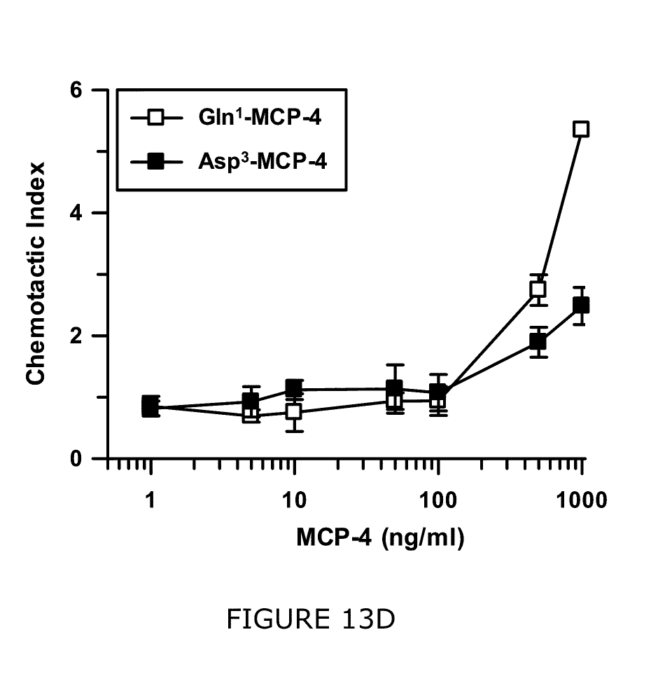

Within the circulation, MCP-1 is protected by a N-terminal pGlu-residue, which confers resistance against N-terminal cleavage by aminopeptidases, e.g. DP4 (FIGS. 3 to 6). As a result of QC inhibitor administration, the unprotected N-terminus is readily cleaved by DP4 (FIG. 7). The N-terminal truncation, in turn, leads to inactivation of human MCP-1 (FIGS. 12 and 13) Taken together, the results imply that the N-terminal pGlu formation represents a mechanism of protection, conferring resistance against N-terminal degradation by post-proline cleaving enzymes, e.g. DP4 and aminopeptidase P (FIG. 5).

Proteolytic Degradation of Human MCP-1(1-76) by Human Serum in Combination with a DP4-Specific Inhibitor

For further investigations on the proteolytic stability of human MCP-1, the data obtained by incubation of MCP-1 with the purified proteases, were substantiated by the incubation of human MCP-1 with human serum. The incubation of human Gln1-MCP-1 with human serum shows the N-terminal truncation of the substrate and the liberation of the first 2 amino acids (Gln1Pro2). In addition, QC activity in plasma competes with the N-terminal proteolysis and stabilizes MCP-1, ending at a final ratio of approx. 60% truncated Asp3-MCP-1 and 40% full-length pGlu1-MCP-1 (FIG. 7A). Furthermore, the pre-incubation of human MCP-1 with human QC leads to the formation of the N-terminal pGlu-residue and, thus, to the stabilization of human MCP-1. At least in the chosen time-frame and dilution of the serum, no degradation of pGlu1-MCP-1 was observed (FIG. 7B). In addition, the incubation of MCP-1 in serum in presence of 9.6 μM of the DP4-inhibitor Isoleucyl-Thiyzolidide also prevents the N-terminal degradation, proving, that MCP-1 is degraded by DP4 or a DP4-like activity in human serum (FIG. 7C).

### Proteolytic Degradation of Human MCP-2, MCP-3 and MCP-4

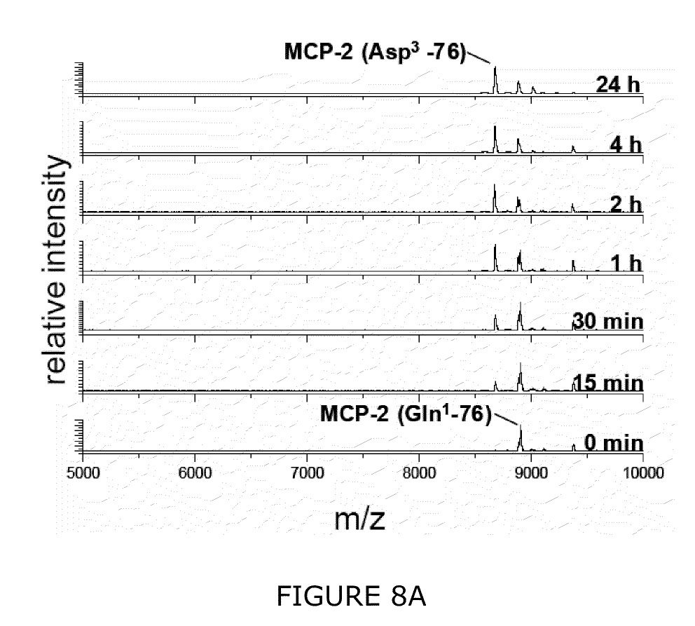

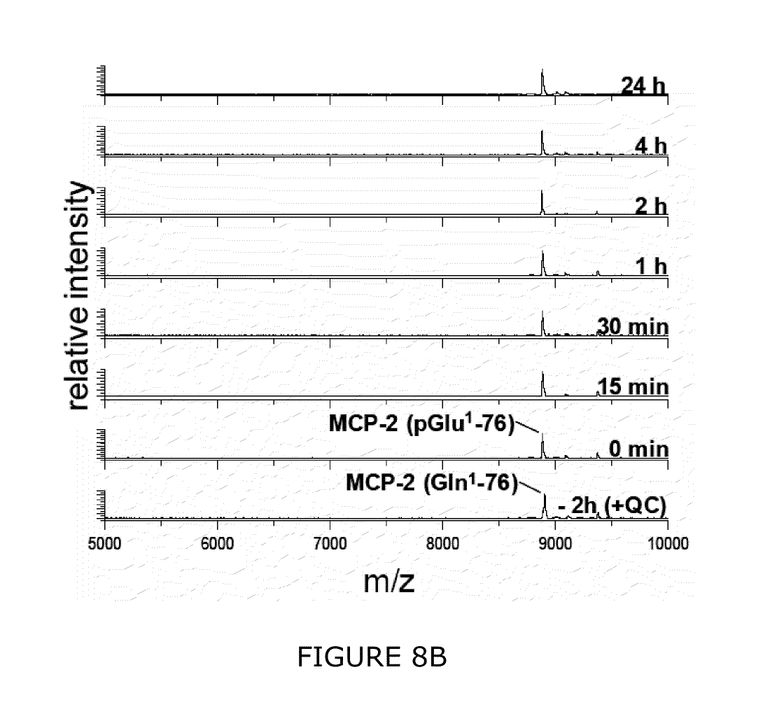

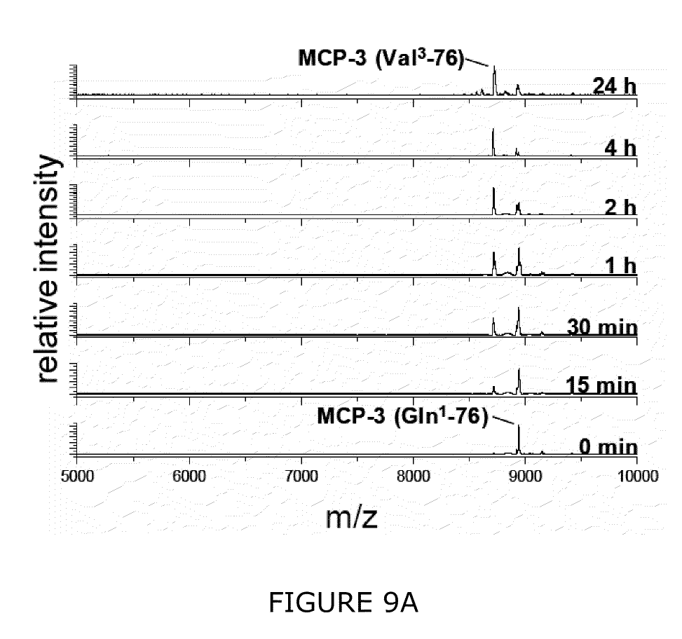

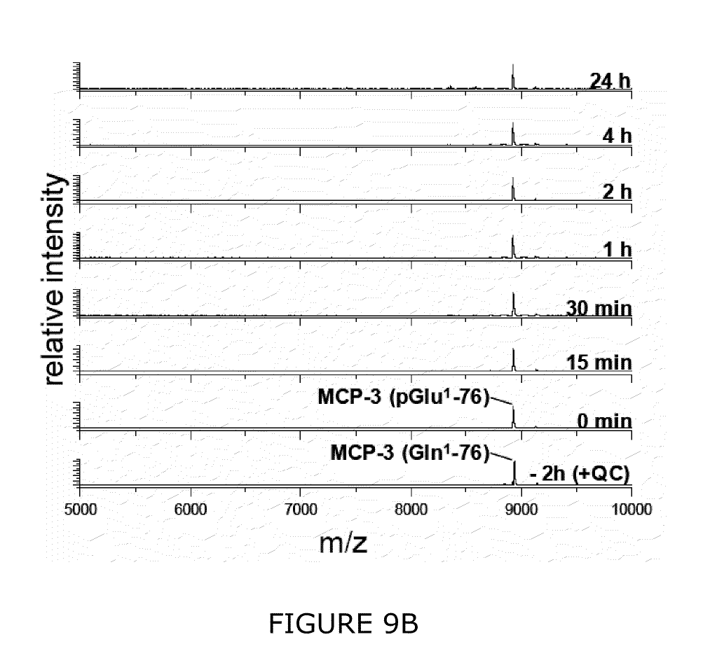

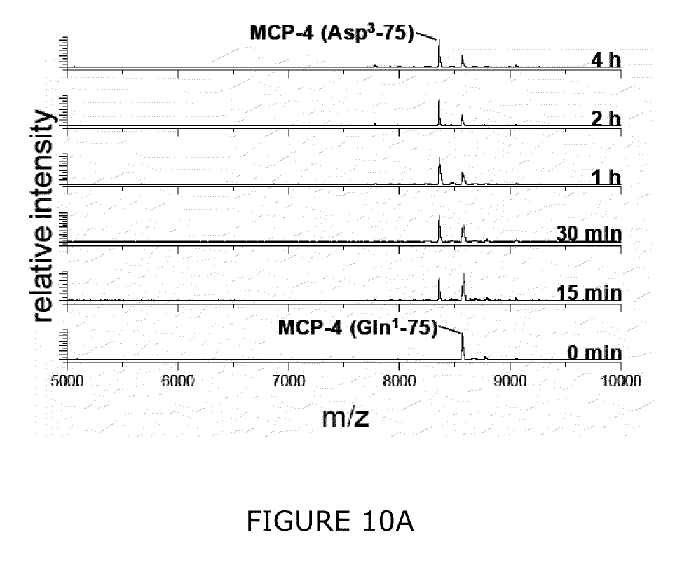

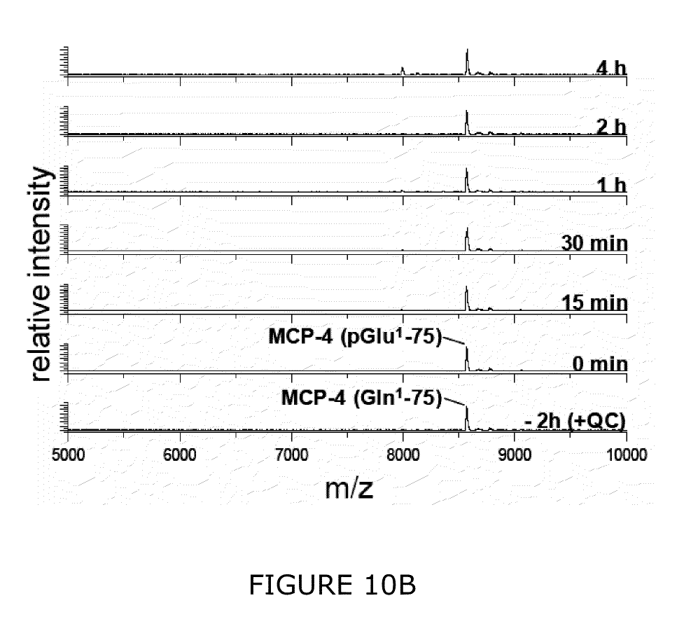

In analogy to the N-terminal degradation of human MCP-1, the susceptibility of other human MCPs, namely MCP-2, MCP-3 and MCP-4, against N-terminal truncation by DP4 was investigated (FIGS. 8 to 10). As observed for MCP-1 before, the N-terminal pGlu-residue protects MCP-2 (FIG. 8B), MCP-3 (FIG. 9B) and MCP-4 (FIG. 10B) against proteolytic degradation by DP4. However, the uncyclized variants, starting with an N-terminal glutamine are readily truncated by DP4 as shown for Gln¹-MCP-2 (FIG. 8A), Gln¹-MCP-3 (FIG. 9A) and Gln¹-MCP-4 (FIG. 10A). Therefore, the N-terminal pGlu-residue stabilizes all MCPs against truncation by aminopeptidases, such as DP4. Thus, the presented concept, to reduce QC activity in vivo in order to provoke accelerated turnover and diminished chemotaxis and receptor activation, applies for all members of the MCP-family.

### Chemotactic Potency of Different N-Terminal Variants of Human MCP-1, MCP-2, MCP-3, MCP-4

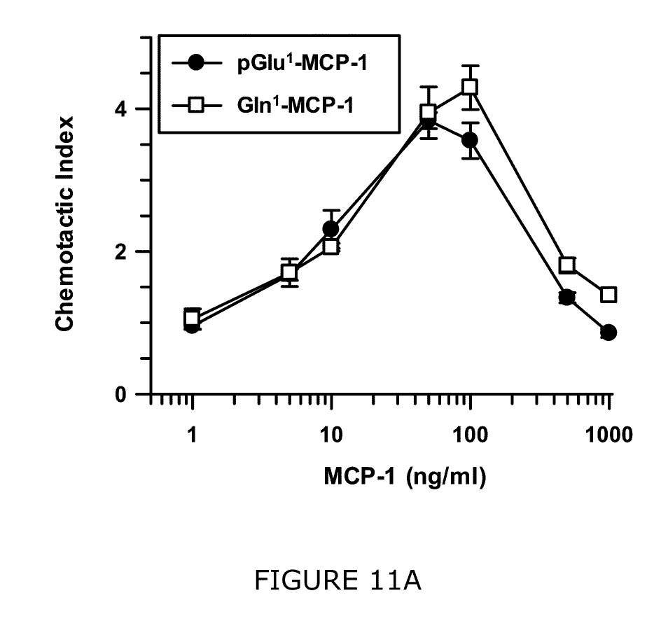

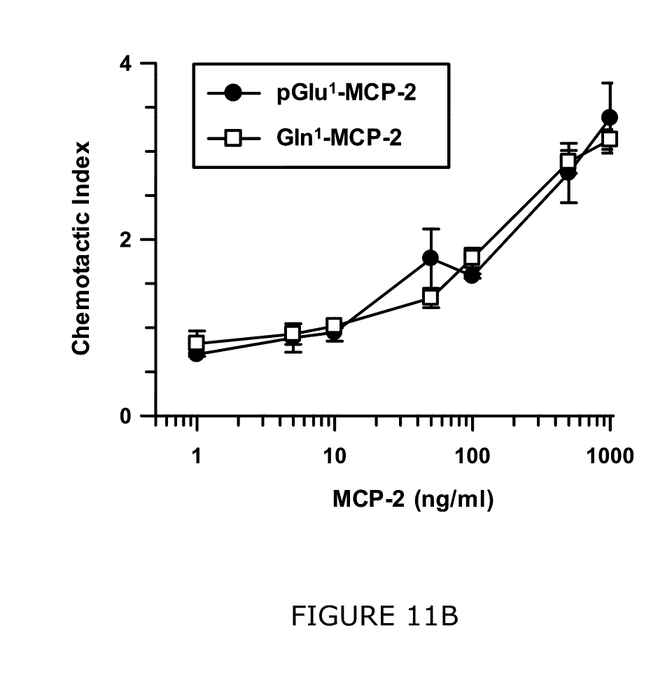

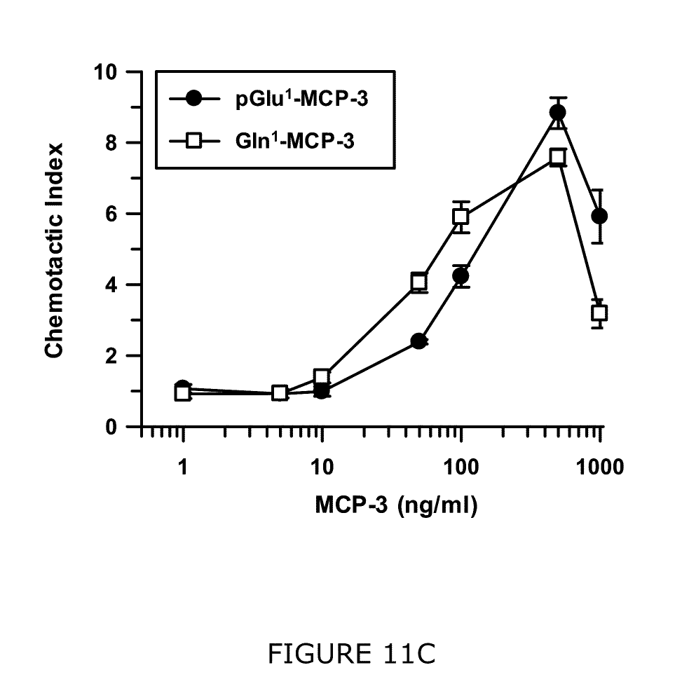

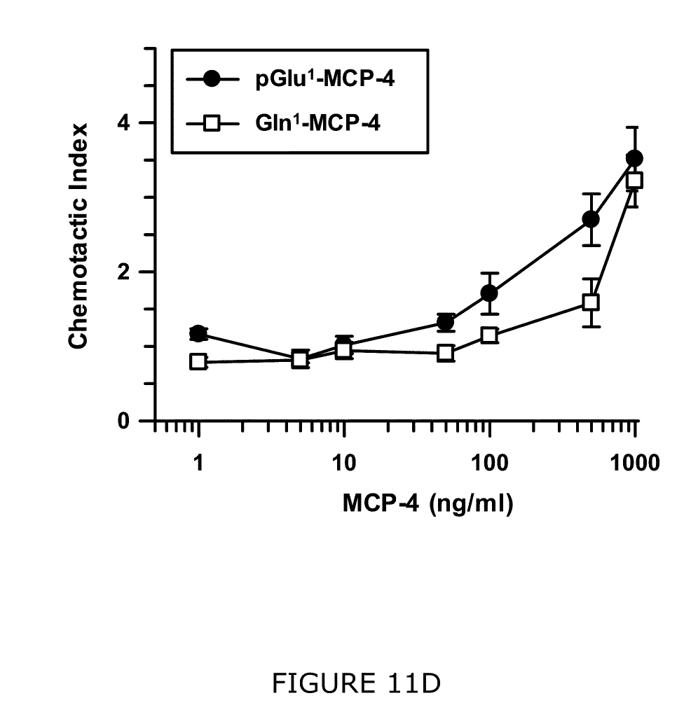

In order to investigate the influence of different N-terminal variants of MCP-1 on the ability to attract human THP-1 monocytes, pGlu¹-MCP-1 and the DP4 cleavage product Asp³-MCP-1 were tested in a chemotaxis assay in vitro. The full-length MCP-1 possessing an N-terminal glutaminyl or pyroglutamyl-residue were found to be equally potent in attracting THP-1 monocytes with a maximum response between 50 ng/ml and 100 ng/ml (FIG. 11A). In addition, the ability of MCP-2, MCP-3 and MCP-4 possessing an N-terminal glutamine or pyroglutamate to attract human THP-1 monocytes was investigated. In analogy to MCP-1, the pGlu-formation at the N-terminus of MCP-2 and MCP-3 has no influence on the potency, compared to the respective glutamine-precursors (FIGS. 11B and C11). However, for MCP-4 the pGlu-formation slightly increases the potency of the peptide (FIG. 11D).

To further investigate the role of QC in stabilizing MCP-1 and its impact on the migration of THP-1 monocytes, Gln¹-MCP-1 was incubated with human DP4. In parallel samples, MCP-1 was pre-incubated with human QC prior to DP4 application. As expected, the obtained dose-response curves imply a proteolytic stability of pGlu¹-MCP-1 reflected by a maximum response at 50-100 ng/ml. In contrast, in absence of QC, Gln¹-MCP-1 is truncated by DP4, which leads to a shift of the dose-response curve to higher MCP-1 concentrations (500-1000 ng/ml) needed to elicit the maximum response. In addition, the pre-incubation of Gln¹-MCP-1 with QC and the QC-inhibitor 1-(3-(1H-imidazol-1-yl)propyl)-3-(3,4-dimethoxyphenyl)thiourea hydrochloride prevents pGlu-formation and, thus, renders the peptide vulnerable to DP4 cleavage, as observed by the shift of the dose-response curve to higher MCP-1 concentrations compared to pGlu¹-MCP-1 (FIG. 12). Therefore, the inhibition of QC leads to the N-terminal destabilization of MCP-1 through degradation by DP4 and, thus, to its inactivation with respect to the monocyte chemotactic activity.

However, since the glutaminyl-precursors are cleaved by DP4 (FIGS. 8A, 9A, 10A), also the potencies of the N-truncated DP4 cleavage products of MCP-2, MCP-3 and MCP-4 were investigated using the chemotaxis assay. For all three variants, the truncation by 2 amino acids leads to a partial inactivation of the chemokines (FIG. 13). Therefore, the pGlu-formation at the N-Terminus of all known MCPs not only protects against N-terminal truncation, but also protects against the loss of chemotactic potency. The presented approach to alleviate the activity of MCP-1 by suppression of N-terminal maturation therefore applies for all members of the MCP family in human beings.

Indirect Sandwich ELISA for the Quantitative Detection of Total Human MCP-1 (hMCP-1) and Human MCP-1 with an N-Terminal Pyroglutamate (hMCP-1 N1pE)

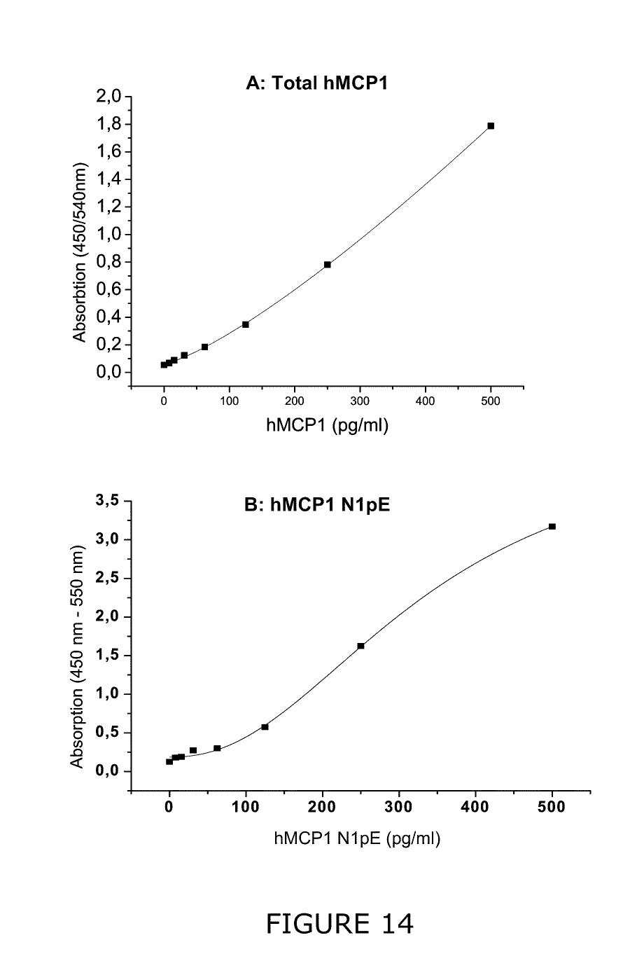

In order to distinguish between both forms and to determine the quantitative amounts of total hMCP-1 and hMCP-1 N1pE in biological samples, we needed to establish two indirect Sandwich ELISAs. FIG. 14 shows two characteristic standard curves for the detection of total hMCP-1 (FIG. 14A) as well as for hMCP-1 N1pE (FIG. 14B).

Evaluation of the Influence of the Standard Peptide Cyclization State to the Total hMCP-1 ELISA

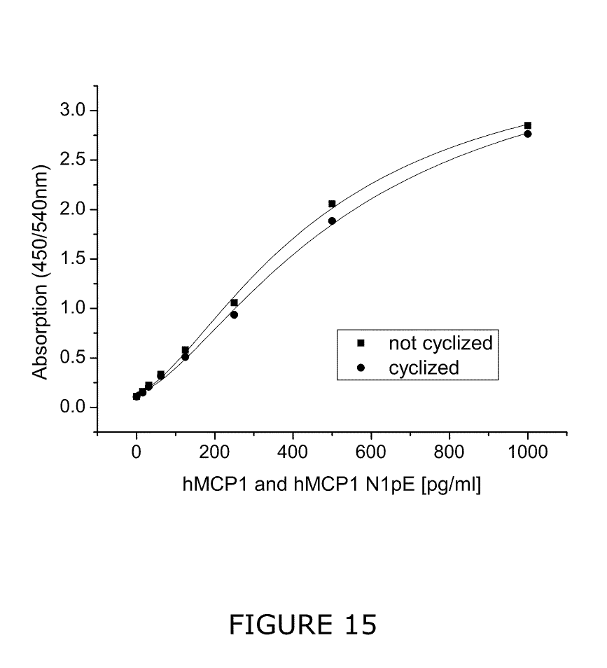

In order to exclude an influence of the cyclization state of the standard peptide to the total hMCP-1 ELISA, we compared the detection of cyclized and not cyclized recombinant human MCP-1 in the same assay (FIG. 15). The experiment reveals no or just a marginal influence of the hMCP-1 peptide cyclization state on its detection in the total hMCP-1 ELISA. This demonstrates that the capture antibody of both ELISAs as well as the detection antibody of the total hMCP-1 ELISA does not interact with the N-terminal amino acid of hMCP-1. This finding is important for the validation of the hMCP-1 and hMCP-1 N1pE ELISAs concerning their ability to determine the ratio of both peptides in samples with varying hMCP-1 N1pE levels.

Determination of the hMCP-1 N1pE/hMCP-1 Ratio in Cell Culture Supernatants of Stimulated NHDF Cells by ELISA

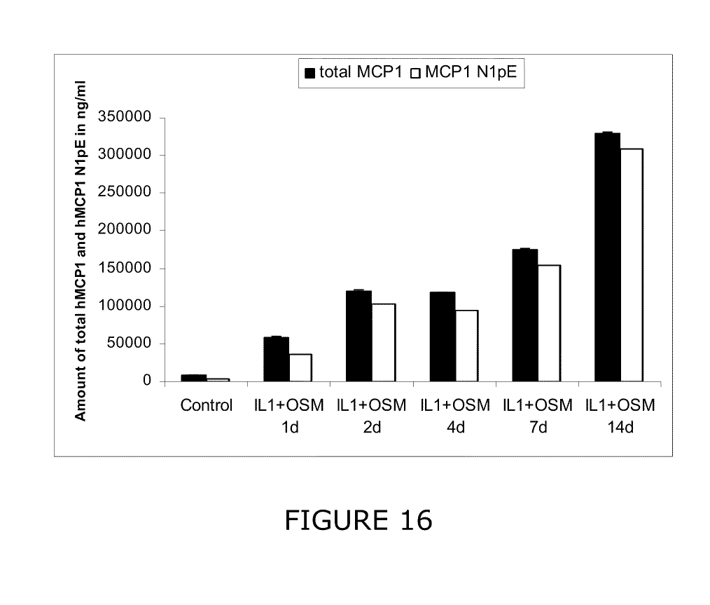

Following an inflammatory stimulus, the expression of hMCP-1 is enhanced in Human Normal Dermal Fibroblasts (NHDF). Hence, the amount of hMCP-1, as well as MCP-1 N1pE, should increase after application of Oncostatin M (OSM) and Interleukin 1β (IL1β) to NHDF. To prove this, OSM and IL1β stimulated NHDF cell culture supernatants were subjected to two ELISA analyses. First, the amount of hMCP-1, and second, the portion of hMCP-1 N1pE were analyzed. The obtained data show (FIG. 16), that the amount of hMCP-1 as well as hMCP-1 N1pE increase in NHDF cell culture supernatant in a time dependent manner following an inflammatory stimulus by OSM and IL1β application. The proportion of hMCP-1 N1pE on the total hMCP-1 level ranges between 70%-95% indicating a near complete N-terminal pyroglutamate modification of the mature MCP-1.

Determination of the hMCP-1 N1pE/hMCP-1 Ratio in Normal Human Dermal Fibroblasts Treated with Glutaminyl Cyclase Inhibitor (QCI)

Since Glutaminyl Cyclase (QC) catalyses the formation of N-terminal pyroglutamate residues, the inhibition of QC should result in decreased hMCP-1 N1pE levels. To prove this, NHDF were stimulated with OSM and IL1β and treated with or without QCI simultaneously.

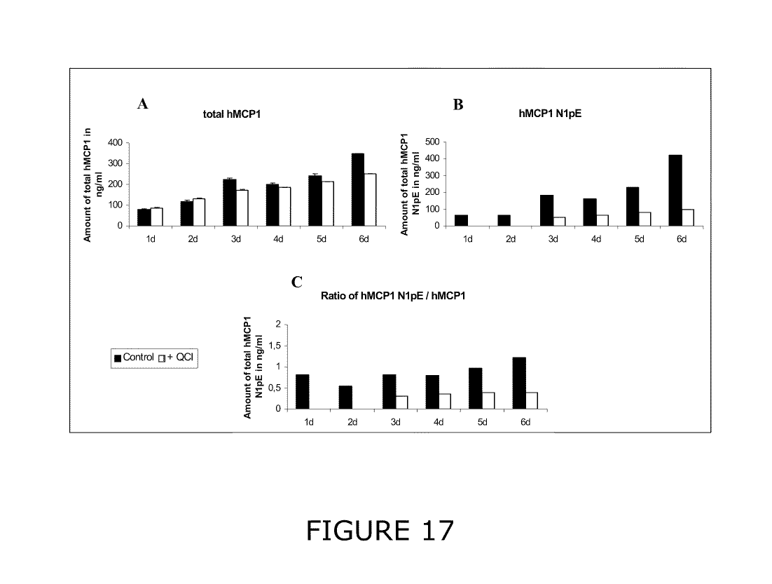

As shown in Example 7 (FIG. 16), the amounts of total hMCP-1 as well as hMCP-1 N1pE increase in a time dependent manner after application of inflammatory cytokines. Addition of QCI results in decreased hMCP-1 N1pE level. Whereas the ratio of hMCP-1 N1pE/hMCP-1 is about 1 in untreated NHDF, QCI treated cells show a ratio of about 0,35. After 1-2 days of OSM+IL1β stimulation, hMCP-1 N1pE level were even below the limit of quantitation (LOQ) of the hMCP-1 N1pE ELISA (see FIG. 17).

Determination of the hMCP1 N1pE/hMCP1 Ratio in a Human Lung Carcinoma Cell Line (A549) Treated with Different Concentrations of QCI

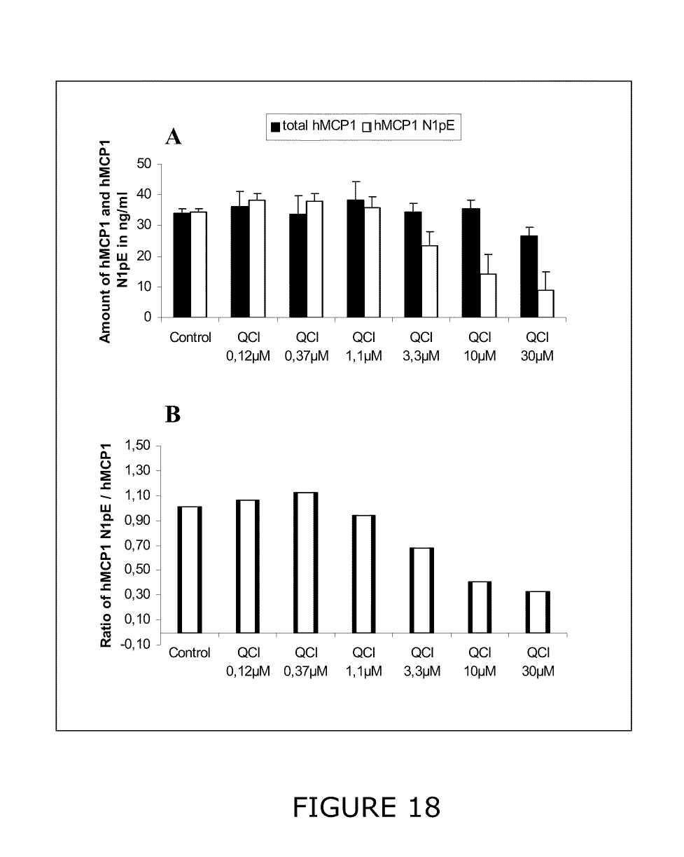

The carcinoma human alveolar basal epithelial cell line A549 was treated with different concentrations of QCI in order to analyze the QCI concentration dependent reduction of hMCP-1 N1pE. The amount of hMCP-1 N1pE is reduced by QCI in a concentration dependent manner whereas the amount of total hMCP-1 is nearly unaffected (FIG. 18). Consequently, the ratio of hMCP-1 N1pE/hMCP-1 decreases with increasing inhibitor concentrations (FIG. 18B).

Spike and Recovery of hMCP-1 and hMCP-1 N1pE in Human Serum

Table 2 shows Spike and Recovery data obtained for hMCP-1 added to human serum. A 66%-81% recovery of the spiked recombinant hMCP-1 peptides was found.

TABLE 2
Spike and Recovery of hMCP-1 in human serum

| Expected Spike | Observed Spike | Observed Spike |
|---|---|---|
| Level of | Level of | Level of |
| hMCP-1[ng/ml] | hMCP-1 [ng/ml] | hMCP-1 in % |
| 6 | 4.87 | 81.09 |
| 3 | 2.28 | 75.88 |
| 1.5 | 1.07 | 71.20 |
| 0.75 | 0.50 | 66.09 |
| 0.38 | 0.28 | 74.20 |

Table 2 shows the expected spike level in comparison to observed hMCP-1 concentrations.

Table 3 shows Spike and Recovery data obtained for the addition of hMCP-1 N1pE in human serum. A recovery of the spiked hMCP-1 N1pE peptides of 66%-79.4% was found.

TABLE 3
Spike and Recovery of hMCP-1 N1pE in human serum.

| Expected Spike | Observed Spike | Observed Spike |
|---|---|---|
| Level of | Level of | Level of |
| hMCP-1 N1pE [ng/ml] | hMCP-1 N1pE [ng/ml] | hMCP-1 N1pE in % |
| 6 | 4.76 | 79.37 |
| 3 | 2.09 | 69.80 |
| 1.5 | 1.05 | 69.81 |
| 0.75 | 0.50 | 66.00 |
| 0.38 | 0.26 | 69.89 |

Table 3 shows the expected spike level in comparison to observed hMCP-1 N1pE concentrations.

These data confirm, that the ELISAs described in Example 5 (FIG. 14) can be used for the quantitative detection of hMCP-1 and hMCP-1 N1pE in human serum. The recovery of spiked peptides is comparable for both ELISAs and fits within an acceptable range.

Establishment of an Indirect Sandwich ELISA for the Quantitative Detection of Total Mouse MCP-1 (mMCP-1) and Mouse MCP-1 with an N-Terminal Pyroglutamate (mMCP-1 N1pE)

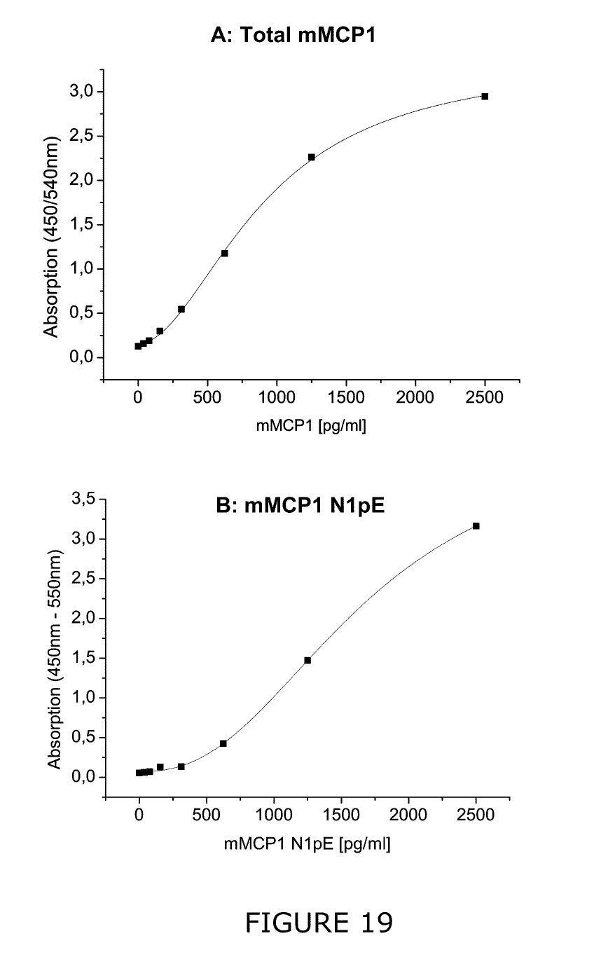

In order to analyse mMCP-1 and mMCP-1 N1pE level in mouse samples, it was necessary to develop assays for the quantification of mouse MCP-1 and mouse MCP-1 N-1pE. Since the MCP-1 N1pE antibody clone 348-2C9 cross reacts with mouse MCP-1 N1pE, this antibody was used for the establishment of an indirect Sandwich ELISA for the detection of mMCP-1 N1pE. Additionally, a comparable indirect Sandwich ELISA was developed to detect total mMCP-1 and to distinguish between both forms of the cytokine. FIG. 19 shows two characteristic standard curves for the detection of mMCP-1 N1pE as well as total mMCP-1.

Evaluation of the Influence of the Standard Peptide Cyclization State on the Total mMCP-1 ELISA

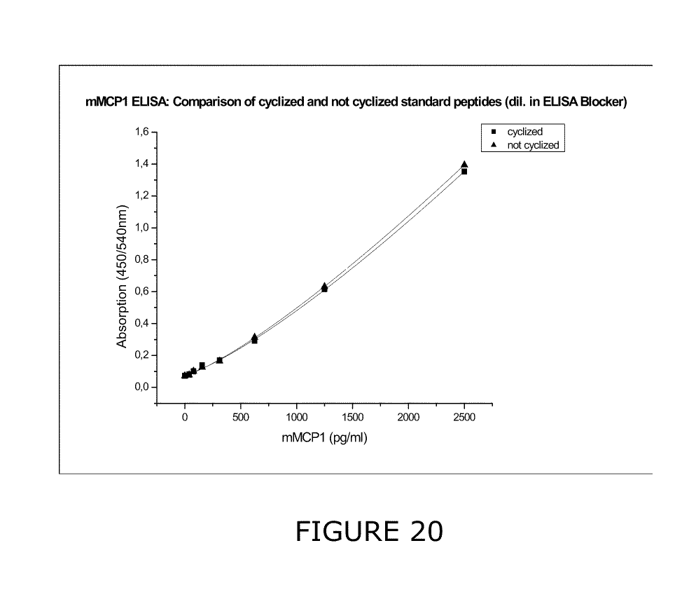

FIG. 20 demonstrates that the mMCP-1 peptide cyclization state did not interfere with the ELISA detection of the total mMCP-1. This ensures the independence of both, the total mouse MCP-1 and the mouse MCP-1 N-1pE ELISA measurements and proves the correctness of the determined mMCP-1 N1pE/mMCP-1 ratio.

Determination of the mMCP-1 N1pE/mMCP-1 Ratio in a LPS Stimulated Murine Macrophage Cell Line RAW 264.7 Treated with Different Concentrations of QCI

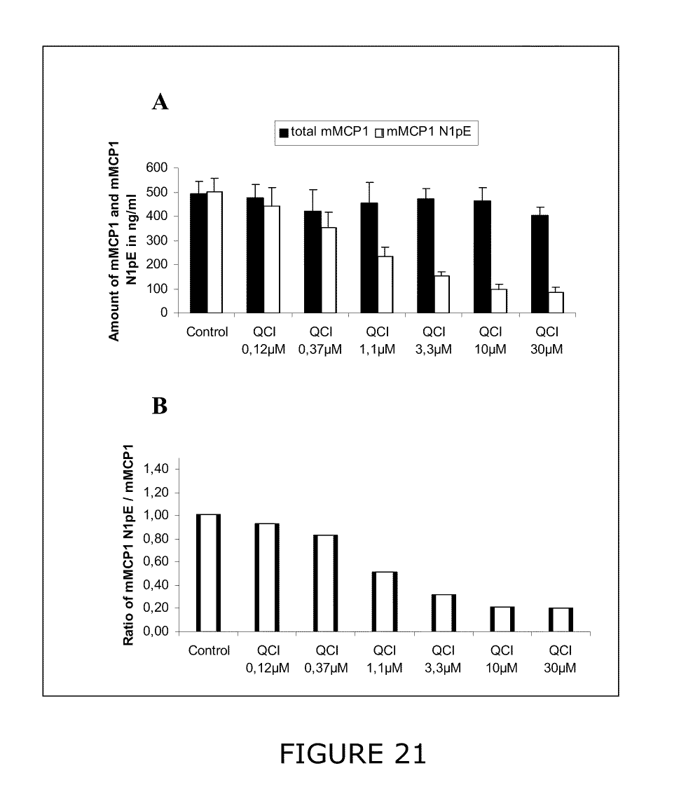

In order to analyse the effect of QCI on the ratio of mouse MCP-1 N1pE/mouse MCP-1, the mouse macrophage cell line RAW 264.7 was stimulated with LPS in the absence or presence of increasing concentrations of the QC inhibitor QCI. The amount of mMCP-1 N1pE is reduced by QCI in a concentration dependent manner whereas the amount of total mMCP-1 remains unaffected. Consequently, analogous to the results in Example 9, the ratio of mMCP-1 N1pE/mMCP-1 decreases with increasing inhibitor concentrations (see FIG. 21).

Cross Validation of mMCP-1 N1pE Level in RAW 264.7 Cell Culture Supernatant by Western Blot Analysis

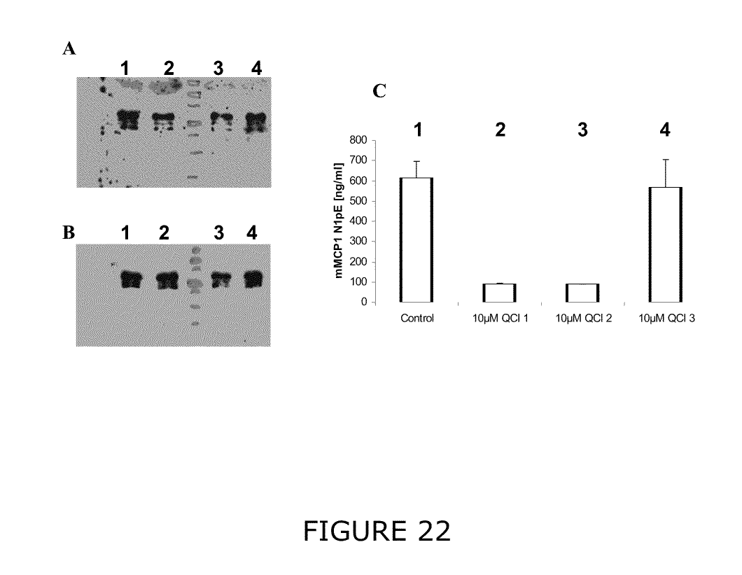

Western Blot analysis was performed to prove that the decreased mMCP-1 N1pE level seen in the ELISA experiments after application of QCI. The experiment was performed using RAW 264.7 cell culture supernatants treated with different QCI. There is no change in the Western Blot signal intensity generated by the antibody Rat anti mouse MCP-1 detecting total mMCP-1 (FIG. 22B). However, the Western Blot signal of mMCP-1 N1pE is concentration dependent (FIG. 22A) and correlates with the corresponding ELISA data (FIG. 22C), showing the different amounts of mMCP-1 N1pE. This experiment demonstrates the correctness of the ELISA data using an alternative assay.

### Measurement of the Binding Affinity of Anti-MCP-1 N1pE Antibody 348/2C9 Compared to Biotinylated MCP-1 N1pE Antibodies Clone 348/2C9

The binding of MCP-1 N1pE antibody 348/2C9 to the antigen hMCP-1(1-38) yields in:

### Stoichiometry: 1.83

Dissociation constant: 151 nM
Reaction enthalpy: −7.679 kcal/mol
Reaction entropy: 5.01 cal/mol·K.

Following the biotinylation reaction, the properties of the derivated antibody has shifted to:

### Stoichiometry: 1.41

Dissociation constant: 444 nM
Reaction enthalpy: −11.44 kcal/mol
Reaction entropy: −9.96 cal/mol·K

The loss of active antibody protein and decrease of affinity was compensated by increase of the antibody concentration used for ELISA experiments (example 16).

Determination of the mMCP-1 N1pE/mMCP-1 Ratio in Fluid Mouse Samples

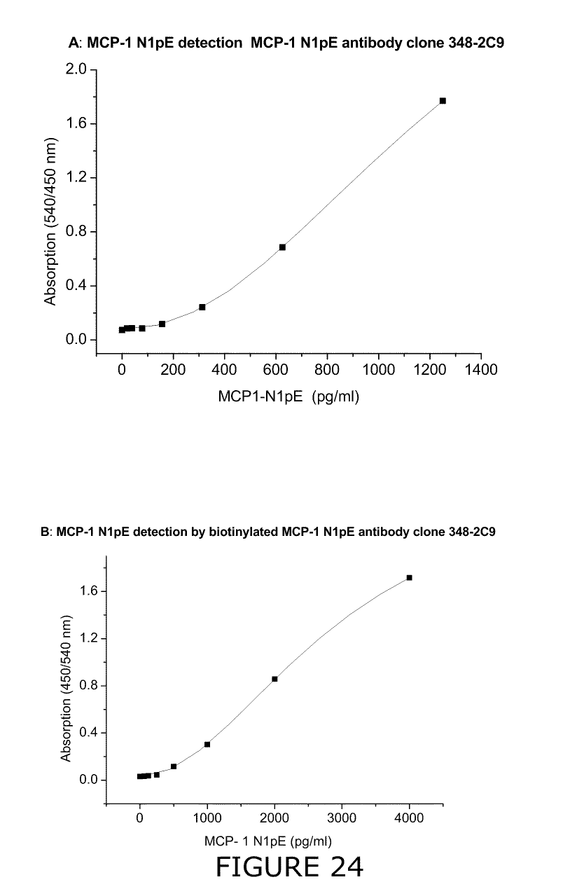

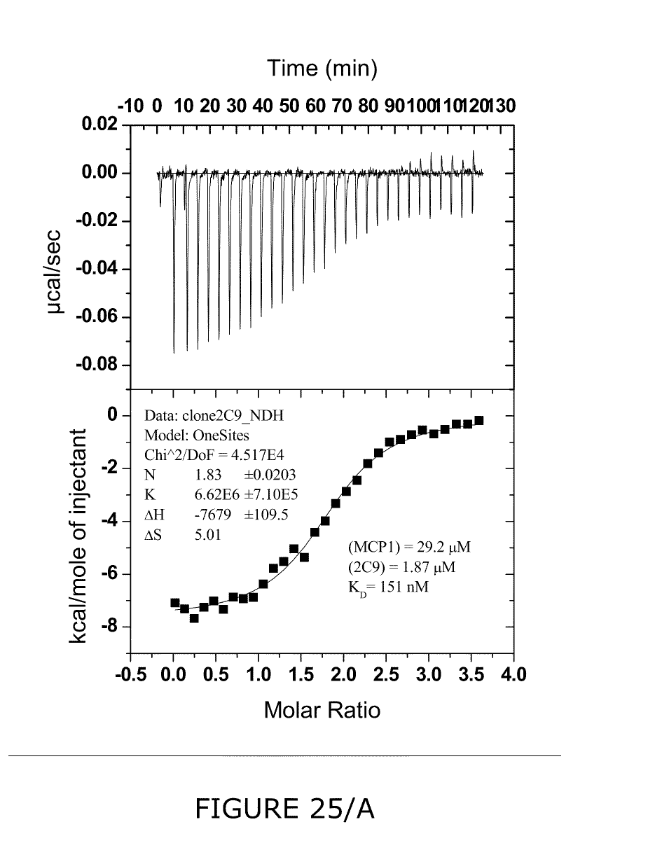

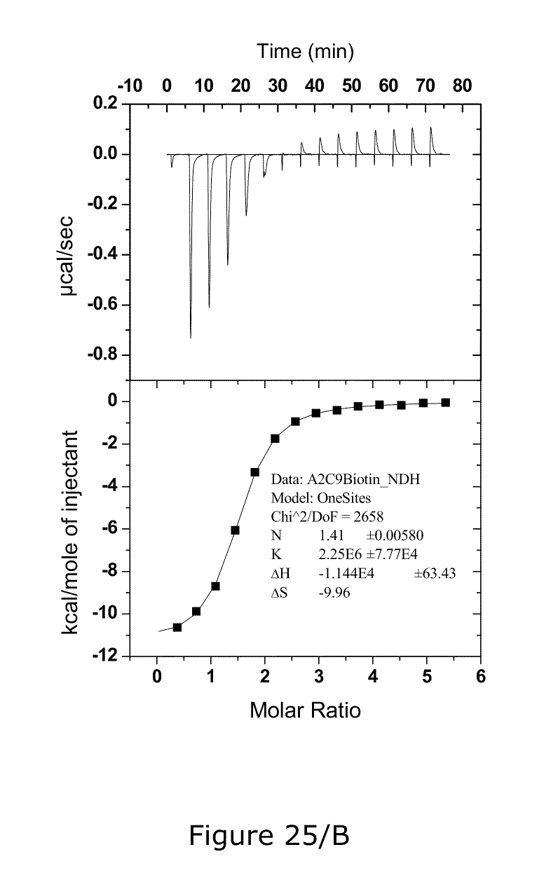

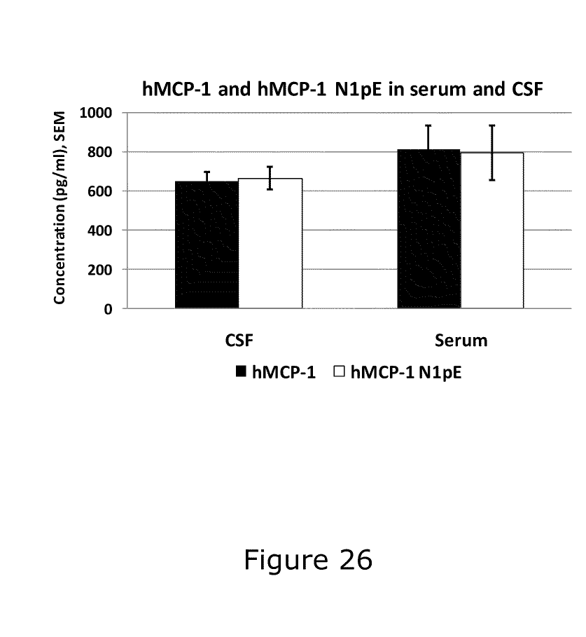

Applying biotinylated MCP-1-N1pE antibody reduce potential cross reactivity of anti-mouse IgG-HRP conjugat against unknown antigens in fluid mouse samples. Biotinylation MCP-1 N1pE antibody resulted in 30% loss of activity (FIG. 25). This can be compensated by increasing the standard peptide concentration to 3000 pg/ml (FIG. 24).

Determination of the mMCP-1 N1pE/mMCP-1 Ratio in Mice Treated with Thioglycollate and Different Concentrations of QCI

To further investigate the effect of QC-inhibitor administration on the ratio of mMCP-1 N1pE/mMCP-1 in vivo QCI was applied to thioglycollate-induced peritonitis.

Beyond the determination of the mMCP-1 N1pE/mMCP-1 ratio, the cellular composition of the peritoneal lavage fluid was determined with special emphasis on infiltrating monocytes (Moma2-positive monocytes/macrophages).

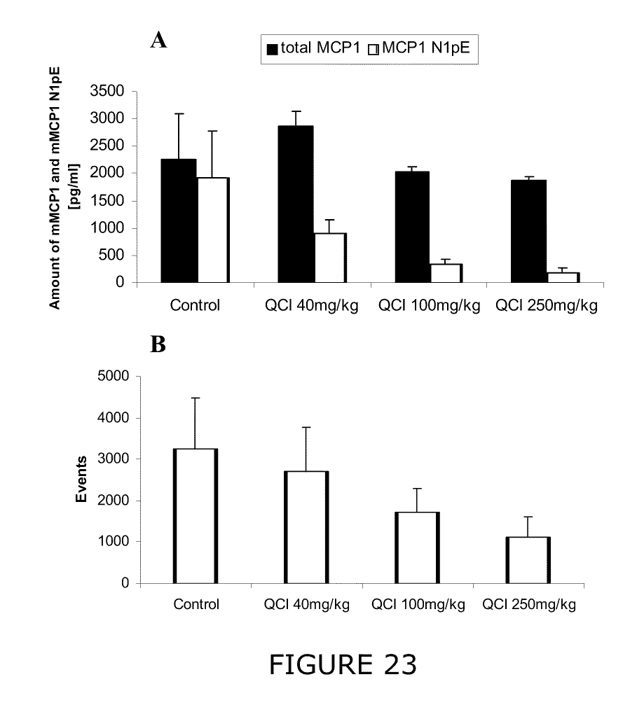

The experiment results in a dose dependent reduction of the mMCP-1 N1pE/mMCP-1 ratio after QCI application (depicted in FIG. 23). Furthermore, the relation of mMCP-1 N1pE level and monocyte invasion into the peritoneum was demonstrated (FIG. 23B). A decreased mMCP-1 N1pE/mMCP-1 ratio results in a decreased number of infiltrating monocytes to the peritoneum.

Such a recruitment of monocytes is a general feature of several inflammatory disorders, for instance, but not limited to pancreatitis, rheumatoid arthritis, atherosclerosis, and restenosis.

The experiment proves the applicability of the MCP-1 N1pE/MCP-1 ratio as biomarker, monitoring the monocytes recruitment capacity of MCP-1. Furthermore the measurement of the MCP-1 N1pE/MCP-1 ratio provides a method for characterization the QC inhibitors' capacity in their application in various inflammatory disorders.

ELISA Measurement of MCP-1 and MCP-1 N1pE in Human CSF and Serum Samples, Derived from 10 Healthy Volunteers

The concentrations of MCP-1 and MCP-1 N1pE were determined on one plate with an intra-assay variation of 1.8% and on two different plates with an intra-assay variation of 2.8%, indicating a great robustness for analysis of human CSF and serum samples. The obtained ELISA signals were 12-times and 6-times above LOQ of total hMCP-1 and hMCP-1 N1pE ELISA, respectively, providing measurement of baseline MCP-1 levels in presence or absence of QC inhibitor to observe treatment or disease related effects.

What is claimed is:

1. A method of diagnosing or monitoring an inflammatory disease or an inflammatory associated disease or of response to treatment thereof, which comprises determining the proportion of N-terminal pyroglutamate modified MCP-1 in relation to the total concentration of MCP-1 within a biological sample.

2. The method according to claim 1, wherein said determination comprises the following steps:
(a) determining a first concentration (cₐ) of N-terminal pyroglutamate modified MCP-1 in a biological sample;
(b) determining a second concentration (c_{d}) of total MCP-1 in said biological sample; and
(d) determining the ratio of cₐ/c_{d}, wherein the value of the first concentration (cₐ) is divided by the value of the second concentration (c_{d}).

3. The method according to claim 2, wherein the ratio of cₐ/c_{d} is 50%, 70% or 85%.

4. The method according to claim 2, wherein step (a) comprises:
i) contacting a biological sample with a capture antibody specific for MCP-1,
ii) application of a detection antibody specific for N-terminal pyroglutamate modified MCP-1,
iii) detection of the resulting immune complex, and
iii) quantifying the captured N-terminal pyroglutamate modified MCP-1 complex.

5. The method according to claim 4, wherein the detection antibody specific for N-terminal pyroglutamate modified MCP-1 comprises a monoclonal antibody produced by a hybridoma cell line selected from the following group:

| | |
|---|---|
| 348/1D4 | (Deposit No. DSM ACC 2905); |
| 348/2C9 | (Deposit No. DSM ACC 2906); |
| 332/4B8 | (Deposit No. DSM ACC 2907); and |
| 332/4F8 | (Deposit No. DSM ACC 2908). |

6. The method according to claim 5, wherein the detection antibody specific for N-terminal pyroglutamate modified MCP-1 comprises a monoclonal antibody produced by a hybridoma cell line selected from 348/2C9 (Deposit No. DSM ACC 2906).

7. The method according to claim 2, wherein step (b) comprises:
i) contacting a biological sample with a capture antibody specific for MCP-1,
ii) application of a detection antibody specific for MCP-1,
iii) detection of the resulting immune complex, and
iv) quantifying the captured MCP-1 complex.

8. The method according to claim 7, wherein the capture antibody specific for MCP-1 comprises an antibody selected from the group consisting of:
polyclonal antiserum goat anti-hMCP1-AF (R&D Systems, Minneapolis, USA);
rabbit polyclonal to MCP-1 antibody ab18072 (Abcam, Cambridge, UK);
rabbit polyclonal to MCP-1 antibody ab9669 (Abcam, Cambridge, UK);
rabbit polyclonal to MCP-1 antibody ab18072 (Abcam, Cambridge, UK);
goat MCP-1 antibody (C-17): sc-1304 (Santa Cruz Biotechnology, Santa Cruz, USA);
polyclonal antiserum rabbit anti mJE (Peprotech, Hamburg, Germany);
rabbit polyclonal to mMCP-1 antibody ab9899 (Abcam, Cambridge, UK);
rabbit polyclonal to MCP-1 antibody ab7202 (Abcam, Cambridge, UK); and
rat monoclonal MCP-1 antibody (JJ5): sc-74215 (Santa Cruz Biotechnology, Santa Cruz, USA).

9. The method according to claim 8, wherein the capture antibody specific for MCP-1 comprises polyclonal antiserum goat anti-hMCP1-AF.

10. The method according to claim 7, wherein the detection antibody specific for MCP-1 comprises an antibody selected from the group consisting of:
mouse anti hMCP-1 (Peprotech, Hamburg, Germany);
mouse monoclonal to MCP-1 antibody ab17715 (Abcam, Cambridge, UK);
mouse monoclonal MCP-1 antibody sc-32819 (Santa Cruz Biotechnology, Santa Cruz, USA);
anti mouse MCP-1 (R&D Systems, Minneapolis, Minn. USA);
hamster monoclonal MCP-1 antibody ab21397 (Abcam, Cambridge, UK);
rat monoclonal MCP-1 antibody ab8101 (Abcam, Cambridge, UK); and
rat monoclonal MCP-1 antibody (JJ5): sc-74215 (Santa Cruz Biotechnology, Santa Cruz, USA).

11. The method according to claim 4, wherein the detection of the complex is carried out by using secondary antibodies, specifically reacting with each detection antibody.

12. The method according to claim 11, wherein the secondary antibodies are anti-mouse antibodies or anti-rabbit antibodies.

13. The method according to claim 12, wherein the secondary antibodies are anti-mouse antibodies.

14. The method according to claim 11, wherein the secondary antibodies are labeled.

15. The method according to claim 14, wherein the secondary antibodies are labelled with horseradish peroxidase (HRP).

16. The method according to claim 4, wherein the detected immune complex is quantified.

17. The method according to claim 4, wherein the captured complexes are quantified by a quantification means selected from the group consisting of: ELISA, such as indirect ELISA, sandwich ELISA, competitive ELISA, reverse ELISA, enzyme-linked immunosorbent spot assay; flow cytometry; Multiplex Assay Systems; immunohistochemistry; immunoprecipitation; and Western Blot analysis.

18. The method according to claim 17, wherein the captured complexes are quantified by a sandwich ELISA as quantification means.

19. The method according to claim 1, wherein the biological sample is selected from the group consisting of blood, serum, urine, cerebrospinal fluid (CSF), plasma, lymph, saliva, sweat, pleural fluid, synovial fluid, tear fluid, bile and pancreas secretion.

20. The method according to claim 19, wherein the biological sample is serum.

21. A method of determining the effectiveness of a glutaminyl cyclase (QC) inhibitor within a biological sample and as a surrogate marker for glutaminyl cyclase (QC) inhibition within a treatment by QC inhibitor application.

22. A method of determining the proportion of N-terminal pyroglutamate modified MCP-1 in relation to the total concentration of MCP-1 within a biological sample which comprises the following steps:
(a) determining a first concentration (cₐ) of N-terminal pyroglutamate modified MCP-1 in a biological sample;
(b) determining a second concentration (c_{d}) of total MCP-1 in said biological sample; and
(c) determining the ratio of cₐ/c_{d}, wherein the value of the first concentration (cₐ) is divided by the value of the second concentration (c_{d}).

23. A method of screening for a glutaminyl cyclase (QC) inhibitor which comprises the steps of:
(a) incubating a control sample comprising MCP-1 and glutaminyl cyclase (QC) and determining the proportion of N-terminal pyroglutamate modified MCP-1 in relation to the total concentration of MCP-1;
(b) incubating a control sample with a mixture comprising MCP-1 and glutaminyl cyclase (QC) together with a glutaminyl cyclase (QC) inhibitor and determining the proportion of N-terminal pyroglutamate modified MCP-1 in relation to the total concentration of MCP-1;
such that a reduction in the ratio of N-terminal pyroglutamate modified MCP-1:total MCP-1 in step (b) relative to step (a) is indicative of glutaminyl cyclase inhibition.

24. A method for measuring the effectiveness of a glutaminyl cyclase (QC) inhibitor which comprises incubating a glutaminyl cyclase (QC) inhibitor with a mixture comprising MCP-1 and glutaminyl cyclase (QC) and determining the proportion of N-terminal pyroglutamate modified MCP-1 in relation to the total concentration of MCP-1.

25. A kit for diagnosing an inflammatory disease or an inflammatory associated disease which comprises a capture antibody specific for N-terminal pyroglutamate modified MCP-1, a capture antibody specific for MCP-1, and instructions to use said kit in accordance with the methods according to claim 1.

26. A method of monitoring efficacy of a therapy in a subject having, suspected of having, or of being predisposed to, an inflammatory disease or an inflammatory associated disease, comprising determining the proportion of N-terminal pyroglutamate modified MCP-1 in relation to the total concentration of MCP-1 in a biological sample from a test subject.

27. The method according to claim 1, which comprises determining the proportion of N-terminal pyroglutamate modified MCP-1 in relation to the total concentration of MCP-1 in a biological sample taken on two or more occasions from a test subject.

28. The method according to claim 27, which comprises comparing the proportion of N-terminal pyroglutamate modified MCP-1 in relation to the total concentration of MCP-1 in the biological samples taken on two or more occasions.

29. The method according to claim 1, wherein the inflammatory disease or inflammatory associated disease is selected from a neurodegenerative disease, chronic and acute inflammation, fibrosis, cancer, metabolic disease, other inflammatory diseases or pathology associated with hyperinsulinemia and obesity.

30. The method according to claim 29 for the detection and diagnosis of atherosclerosis, rheumatoid arthritis, asthma, delayed hypersensitivity reactions, pancreatitis, Alzheimer's disease, lung fibrosis, renal fibrosis, gestosis, graft rejection, neuropathic pain, diabetic nephropathy, colitis, stroke, AIDS and tumors.

31. The method according to claim 29 for the detection and diagnosis of Alzheimer's disease, atherosclerosis, rheumatoid arthritis, restenosis and pancreatitis.

32. The method according to claim 29 for the detection and diagnosis of Alzheimer's disease or rheumatoid arthritis.
